(19) United States
(12) Patent Application Publication
(10) Pub. No.: US 2012/0088968 A1
(43) Pub. Date: Apr. 12, 2012

(54) Methods and devices for visualization and access

(75) Inventors: Kabir GAMBHIR, San Diego, CA (US); Thomas A. RITTER, San Marcos, CA (US)

(73) Assignee: EPIC MEDICAL INC., San Clemente, CA (US)

(21) Appl. No.: 13/159,295

(22) Filed: Jun. 13, 2011

Related U.S. Application Data
(63) Continuation-in-part of application No. 12/902,131, filed on Oct. 11, 2010.

(51) Int. Cl. A61B 1/018

(52) U.S. Cl. 600/106; 600/104

(57) ABSTRACT

Methods and devices for visualizing and accessing a region inside a body are described. One embodiment of a device includes a working catheter which slides along an open track in a visualization catheter. The visualization catheter is inserted into the body to locate a region of the body with the aid of the visualization element. The working catheter then slides along the track to reach the region, and a working element is inserted through the working catheter to access the region, again with the aid of the visualization element. The methods and devices may also be used to access a variety of internal cavities, soft tissues and organs, and the mediastinal space.

### CROSS-REFERENCE TO RELATED APPLICATIONS

This application is a continuation-in-part of U.S. patent application Ser. No. 12/902,131, filed Oct. 11, 2010.

### BACKGROUND

The pericardium is a tough, fibrous sac which surrounds and protects the heart. The pericardial space is formed between the two layers of the pericardium, the parietal pericardium and the serous pericardium. The serous pericardium has two layers, the first a fibrous layer and the second the epicardium which is closest to the heart. Pericardial fluid within the pericardial space serves to lubricate the motion of the heart.

The pericardial space may be accessed to treat the heart for any one of a number of conditions. For example, the pericardial space may be accessed to perform epicardial ablations for the treatment of arrhythmias such as atrial fibrillation. The pericardial space may also be accessed to deliver drugs and stem cells for the treatment of heart attacks.

The pericardial space may be accessed using minimally invasive techniques. One common technique involves guiding a needle to the pericardium, and then advancing the needle through the pericardium, all under fluoroscopy. However, because of anatomical variations and previous procedures, it may take up to an hour to navigate less than 10 cm through the body to locate a suitable area on the pericardium to create an access site. Navigating through the body with a sharp needle creates the risk of causing damage to structures such as the liver. During pericardial access, the risk posed by a sharp needle may cause damage to the underlying structures such as the coronary arteries and myocardium.

The mediastinal space is the region between the two pleural sacs, with the sternum in front and the vertebral column behind. The mediastinal space can be an especially difficult area to access, especially in the area posterior of the heart, superior to the diaphragm, and inferior to the clavicle.

What is needed are methods and devices which will reduce the amount of time needed to locate the pericardium and other regions inside the body, and reduce the risk of unintended puncture or damage to other structures during the location process.

What is also needed are methods and devices which will facilitate the creation of an access site through the pericardium and other regions inside the body, while reducing the risk of damage or irritation to underlying structures.

What is also needed are methods and devices which will facilitate access to the mediastinal space and other regions inside the body.

### SUMMARY

In one embodiment, an access device comprises a visualization catheter, a visualization element coupled to a distal end of the visualization catheter, and an open track formed along a length of the visualization catheter. The access device also comprises a working catheter configured to slide along the track until a distal end of the working catheter is in a vicinity of the visualization element, and a working lumen extending through the working catheter.

### BRIEF DESCRIPTION OF THE DRAWINGS

FIGS. 1A-1D shows one embodiment of an access device 100.

FIGS. 2A-2F show one method of using access device 100.

FIGS. 3A-3D show another embodiment of an access device 200.

FIGS. 4A-4G show one method of using access device 200.

FIGS. 5A-5D show yet another embodiment of an access device 300.

FIGS. 6A-6G and 7A-7D show one method of using access device 300.

FIGS. 8A-8C show other embodiments of a distal portion of access device 300.

FIGS. 9A-9D show still another embodiment of an access device 400.

FIGS. 10A-10C show another embodiment of an access device 500. FIGS. 10D-10E show alternative embodiments of access device 500.

FIGS. 11A-11H show one method of using access device 500.

FIGS. 12A-12C show another embodiment of an access device 600. FIGS. 12D-12E show alternative embodiments of access device 600.

FIGS. 13A-13H show one method of using access device 600.

FIGS. 14A-14B show one embodiment of an imaging package 700.

FIGS. 15A-15F show one method of assembling imaging package 700.

### DETAILED DESCRIPTION

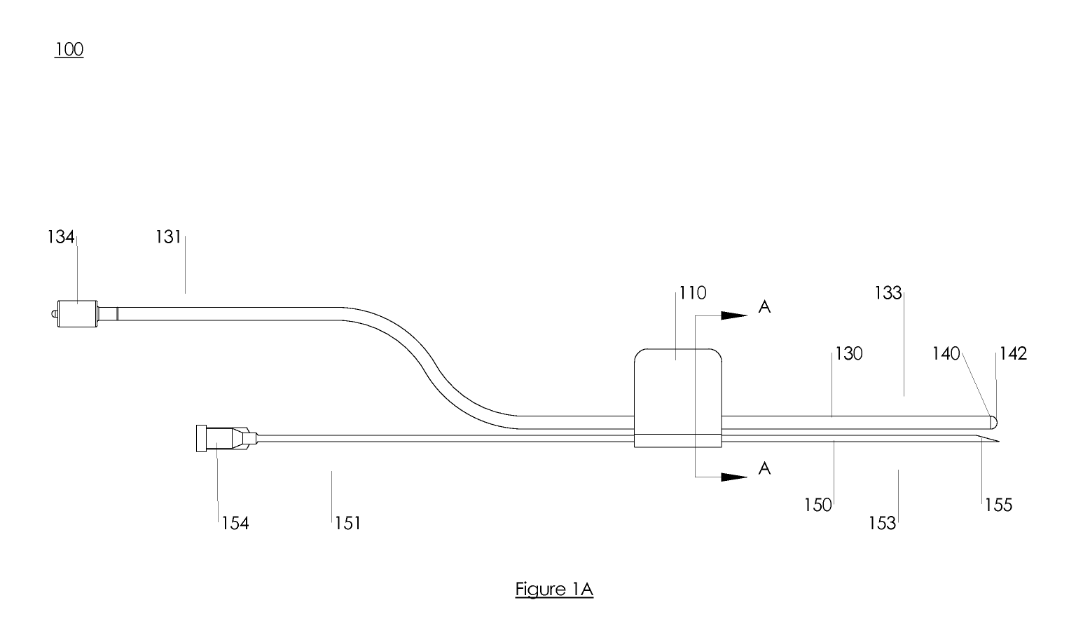

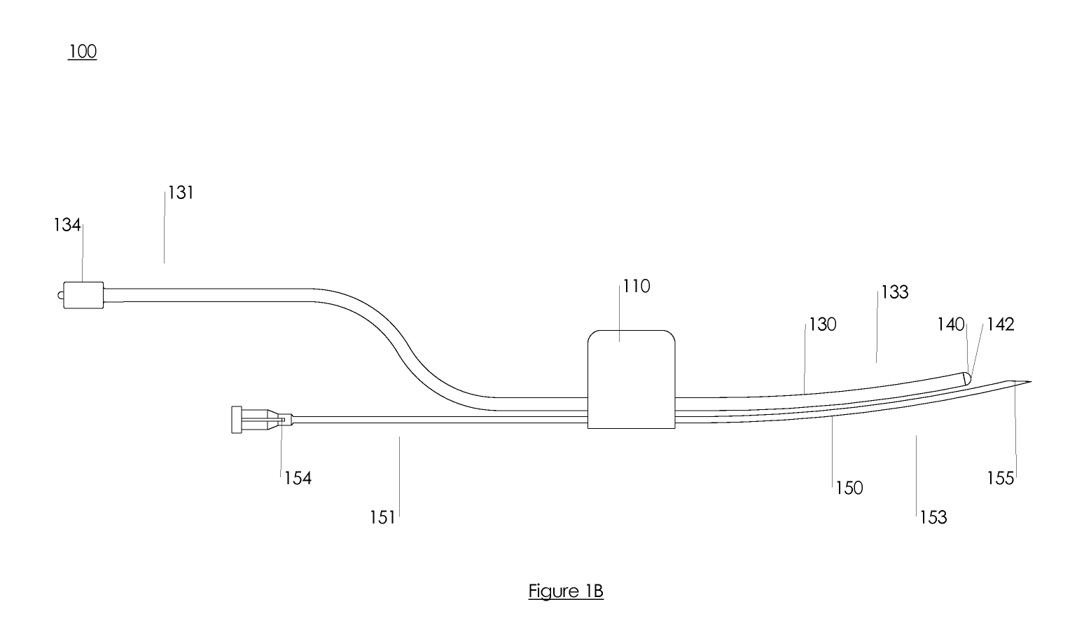

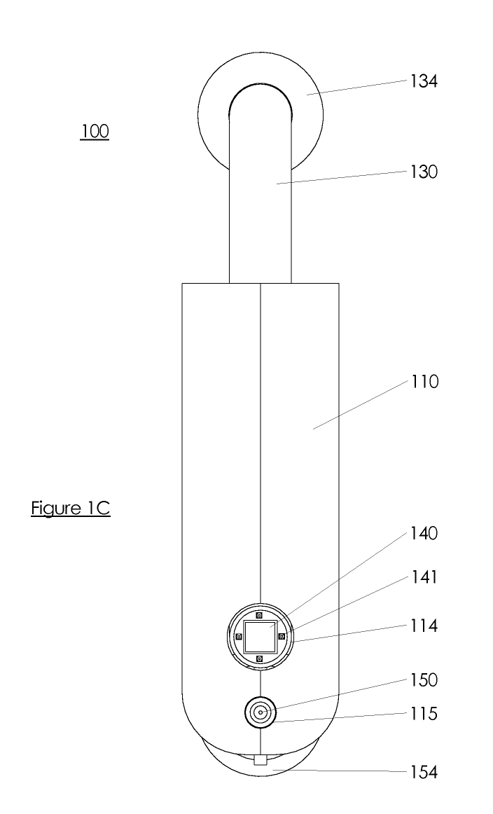

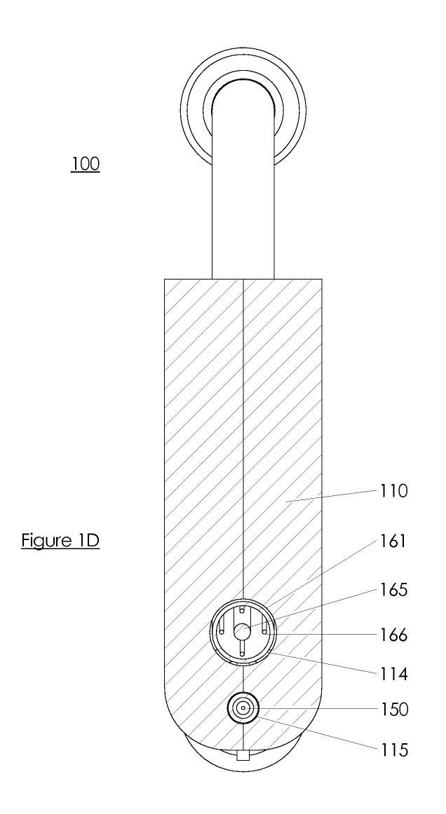

FIGS. 1A-1B show side views of one embodiment of an access device 100. FIG. 1C shows an end view of access device 100. FIG. 1D shows a cross-sectional end view of access device 100.

Access device 100 includes a handle 110, a visualization catheter 130 with a visualization element 140, and an access element 150.

Handle 110 includes a catheter lumen 114 and an access lumen 115. Handle 110 may be constructed as two halves or as a clamshell.

Visualization catheter 130 is at least partially positioned within catheter lumen 114, and can slide and rotate within catheter lumen 114. Visualization catheter 130 includes a proximal portion 131 and a distal portion 133. Visualization catheter 130 may be a hollow tube made of a ductile material such as stainless steel or any other suitable material. Visualization catheter 130 includes a lumen 161. Proximal portion 131 may be configured to facilitate rotation of visualization catheter 130 within catheter lumen 114. Proximal portion 131 may be configured with an S-shaped bend to facilitate manipulation of visualization catheter 130. Proximal portion 131 may include a coupling 134 for attachment of a power source and a video monitor. Distal portion 133 includes a visualization element 140 and one or more lights 141.

Visualization element 140 and lights 141 may be coupled to the tip or end of distal portion 133. Alternatively, visualization element 140 and lights 141 may be coupled to the side or any other suitable location of distal portion 133. Visualization element 140 and lights 141 are coupled to visualization wires 165 and light wires 166 which pass through lumen 161 to coupling 134. Visualization element 140 and lights 141 are covered by a lens 142. Lens 142 may have a hydrophobic coating or other coating to reduce adhesion of natural and synthetic materials that would obscure the image. As shown in FIG. 1B, distal portion 133 may have a curved configuration, and may be bent or otherwise configured by the user and hold its shape.

Access element 150 is at least partially positioned within access lumen 115, and can slide and rotate within access lumen 115. Access element 150 may be used for injection of a liquid, passing of a guidewire 105, application of a vacuum, or any other suitable purpose. Access element 150 includes a proximal portion 151 and a distal portion 153. Proximal portion 151 may include a coupling 154. Distal portion 253 has a tip 255 that may be a blunt tip trocar, a blunt tip obturator, a sharp edge trocar, a sharp edge needle (e.g., Tuohy, epidural, biopsy), a guidewire tip, or any other suitable instrument. Access element 150 may be configured to work with an RF, microwave, cryoablation, high intensity focused ultrasound (HIFU), laser, or any other suitable energy source. Distal portion 153 may have depth markings. Distal portion 153 may be connected to an ohmmeter to measure impedance as the needle penetrates the pericardial membrane into the pericardial space. The impedance measurement may be used to provide an indication as to whether the pericardial membrane has been penetrated. As shown in FIG. 1B, distal portion 153 may have a curved configuration, and may be bent or otherwise configured by the user and hold its shape.

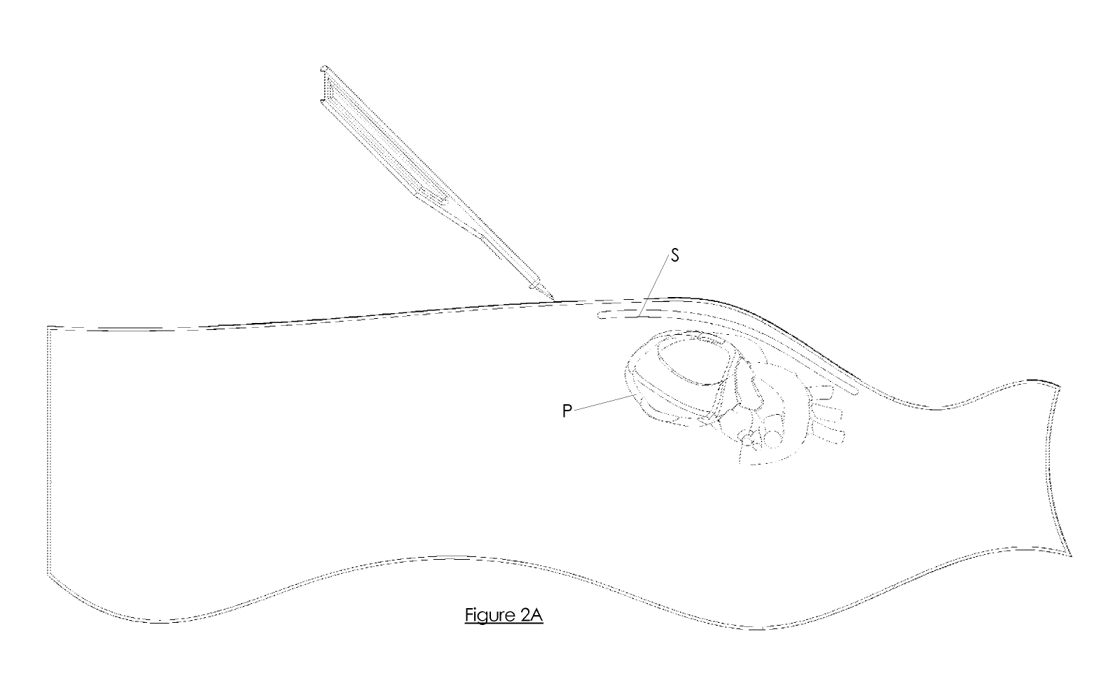

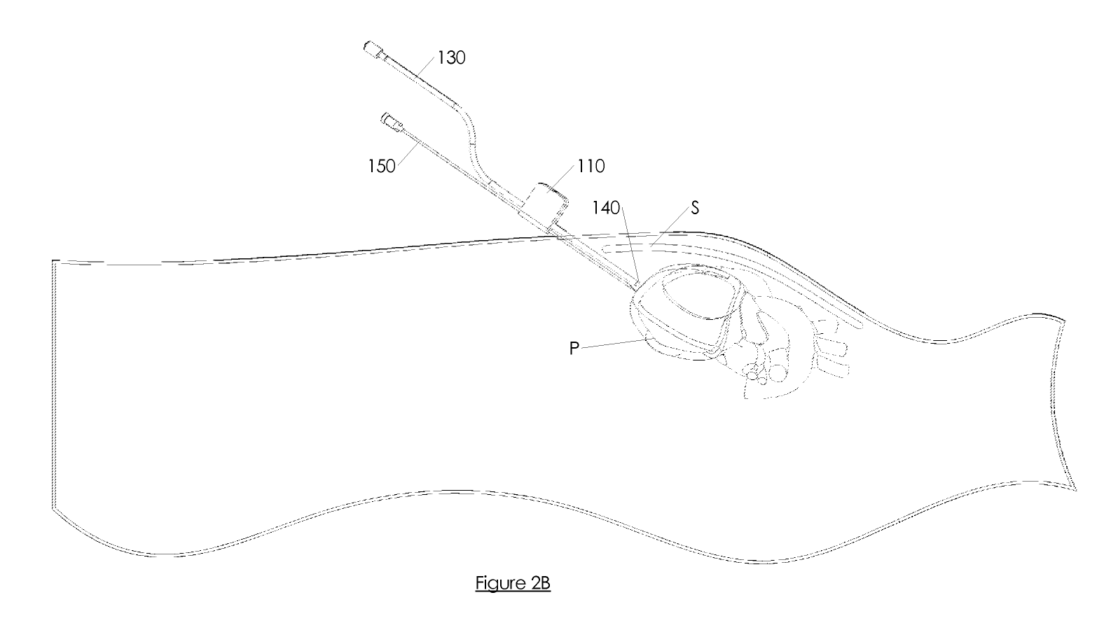

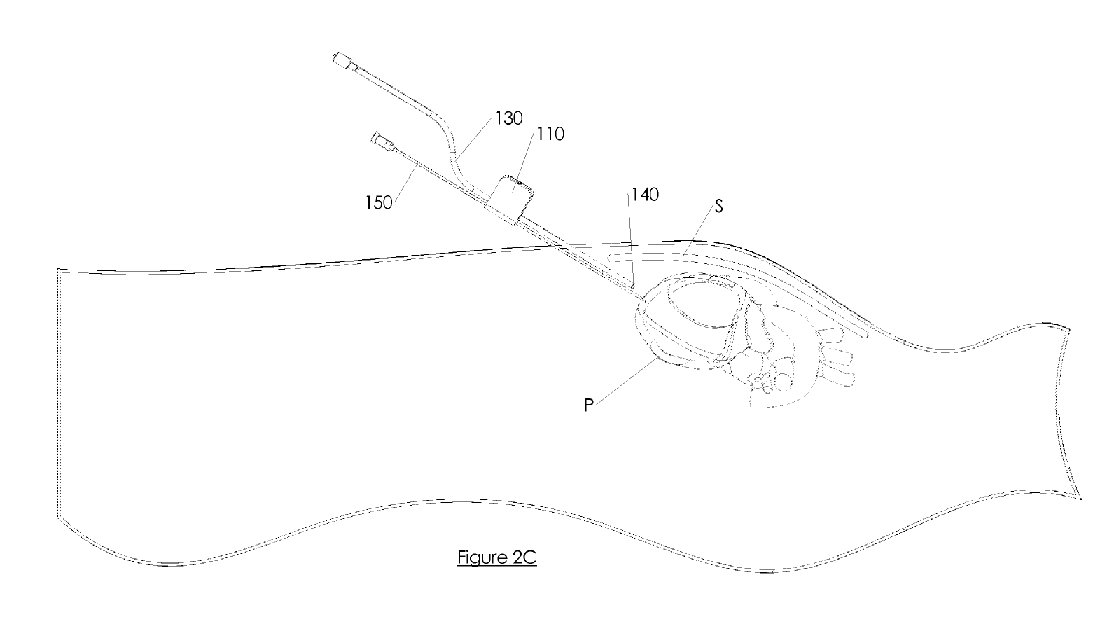

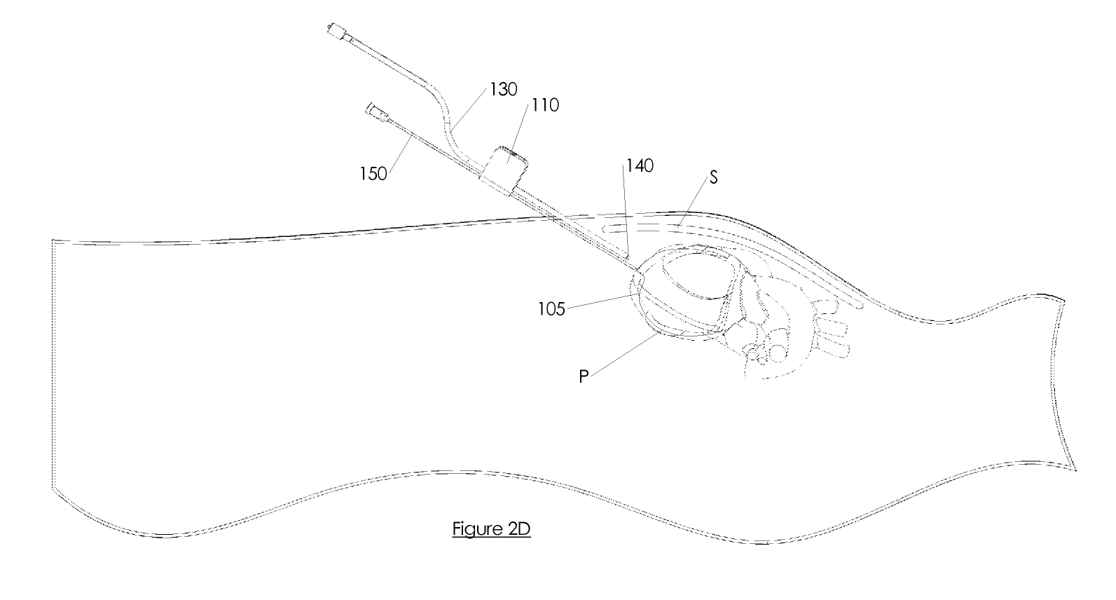

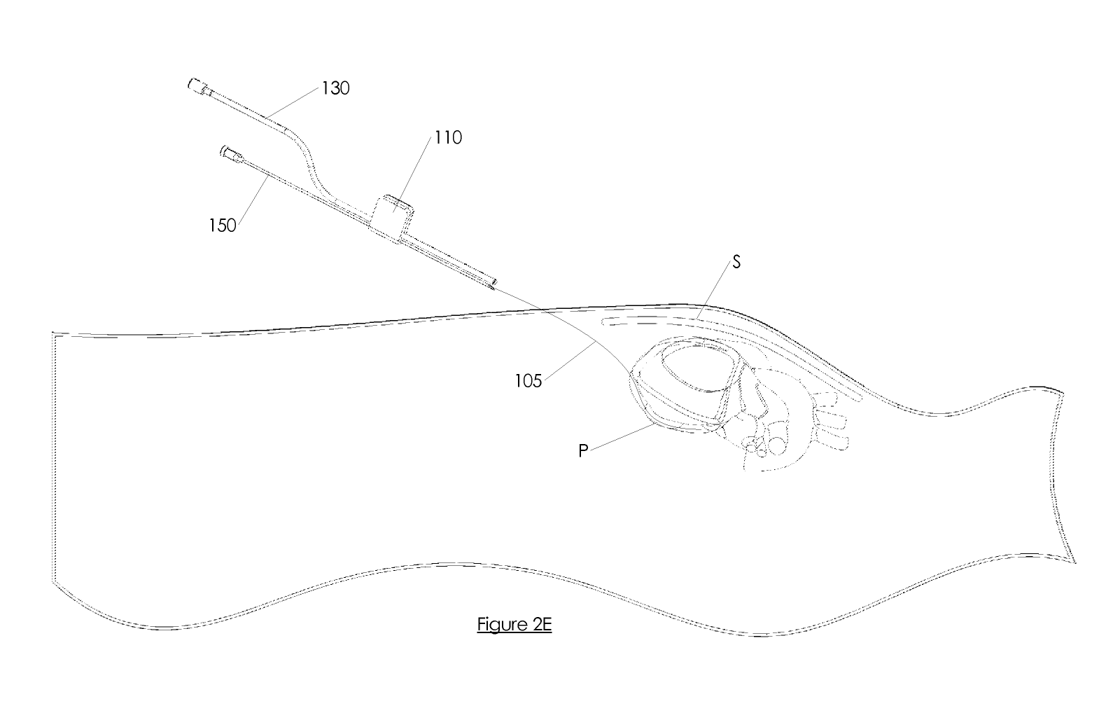

FIGS. 2A-2F show one method of using access device 100.

FIG. 2A shows a percutaneous puncture being made for a subxiphoid approach. Alternatively, an intercostal, apical, subclavian, suprasternal, or any other suitable approach may be used.

FIG. 2B shows visualization catheter 130 and access element 150 inserted through the puncture and positioned at or near the surface of the pericardium P. Visualization element 140 is used to guide visualization catheter 130 and access element 150 along the posterior aspect of the sternum S to the surface of the pericardium P. Visualization catheter 130 may be rotated and moved in and out.

FIG. 2C shows access element 150 advanced through pericardium P to create an access site. For an access element 150 having a sharp tip 155, visualization element 140 may be used to visualize access element 150 as it is advanced through pericardium P. For an access element 150 used with RF energy, visualization element 140 may be used to visualize access element 150 as RF energy is passed through access element 150 to penetrate pericardium P. Access element 150 may be rotated so that a desired surface is visible to visualization element 140. Saline, contrast, medications, and/or other fluids may be introduced through access element 150 into the pericardial space.

FIG. 2D shows guidewire 105 passed through access element 150 and positioned in the pericardial space.

FIG. 2E shows visualization catheter 130 and access element 150 withdrawn, leaving guidewire 105 in place.

FIG. 2F shows a sheath 180 advanced over guidewire 105 through the puncture and the access site and into the pericardial space. Other devices or guidewires may be advanced through sheath 180 to access the pericardial space. Saline, contrast, medications, and/or other fluids may be introduced through sheath 180 into the pericardial space.

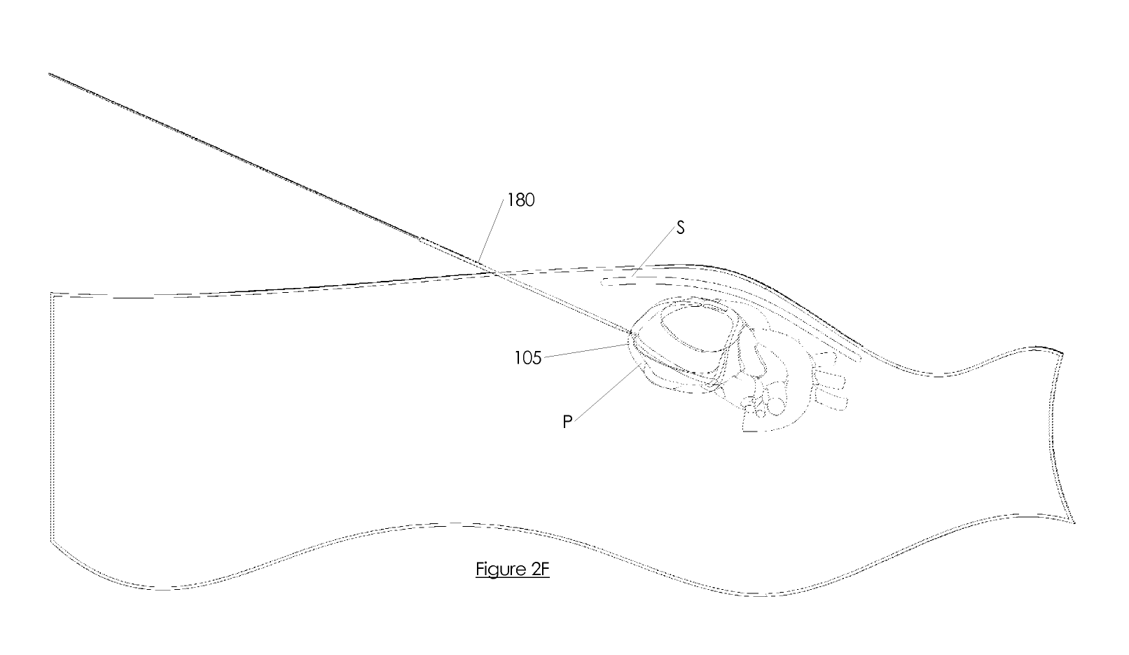

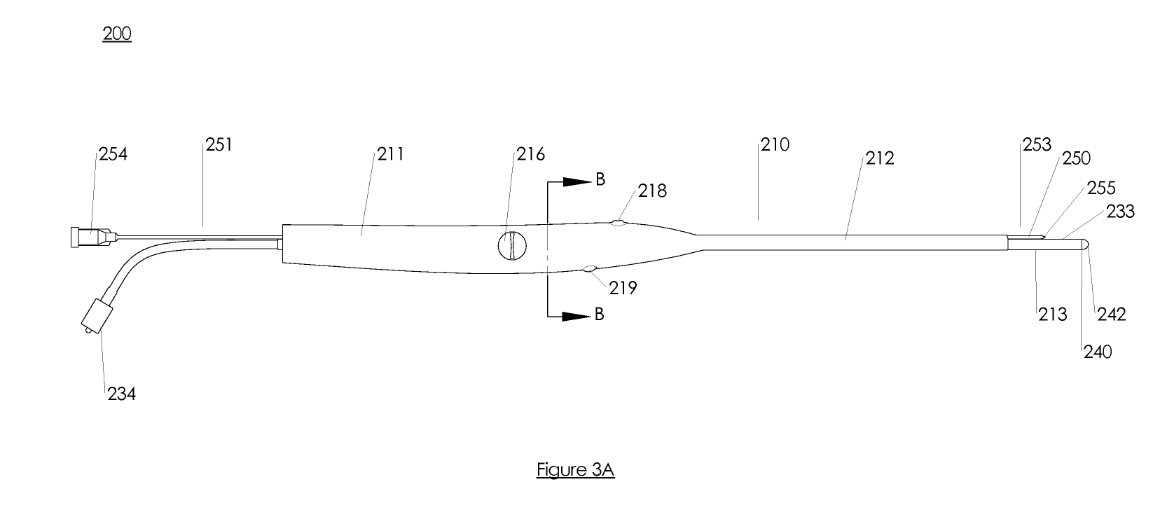

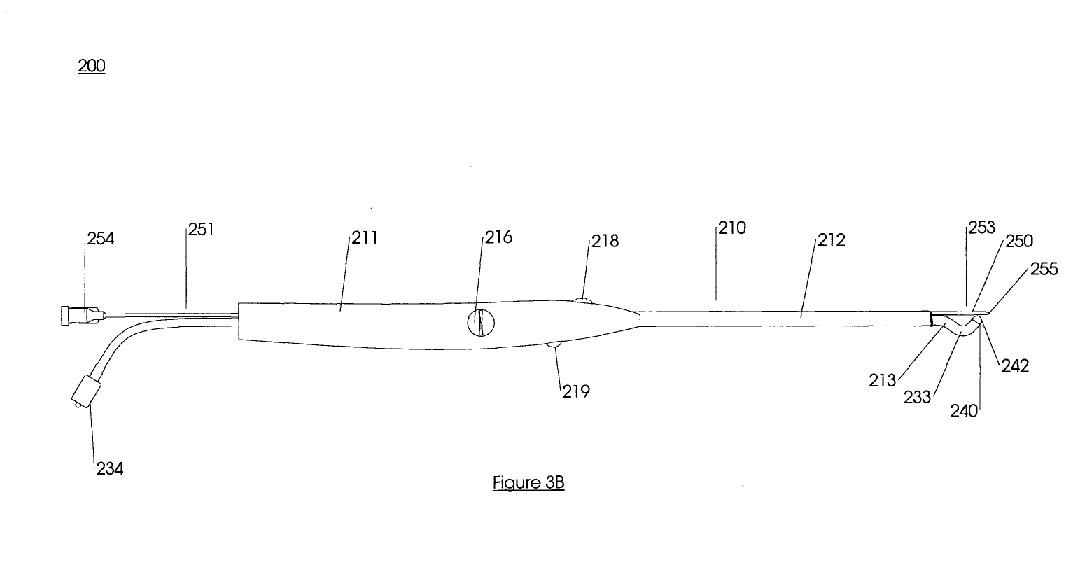

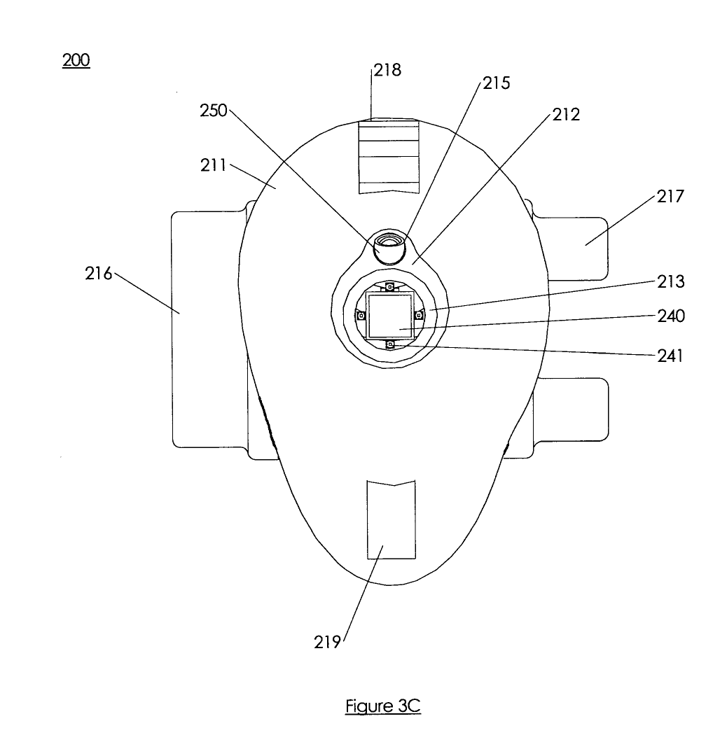

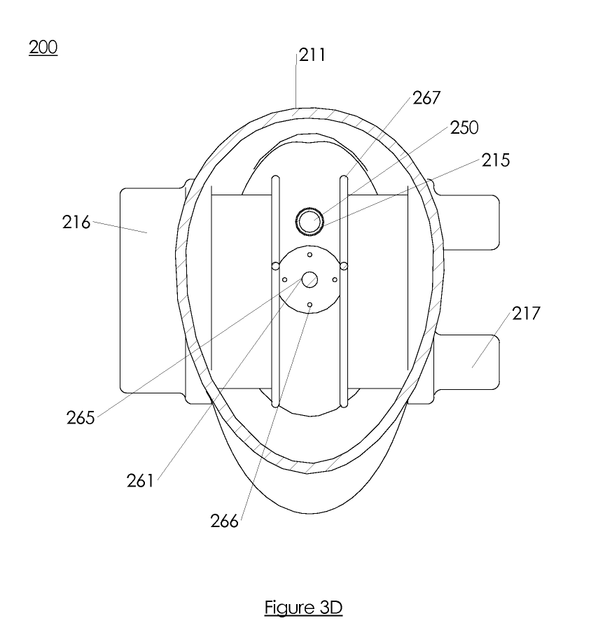

FIGS. 3A-3B show side views of another embodiment of an access device 200. FIG. 3C shows an end view of access device 200. FIG. 3D shows a cross-sectional end view of access device 200.

Access device 200 includes a housing 210, a visualization element 240, and an access element 250.

Housing 210 includes a handle 211, a central portion 212, and a deflectable portion 213. Housing 210 also includes an access lumen 215 and a visualization lumen 261. Handle 211 includes a steering control 216, a tension lock 217, a visualization control 218, and a light control 219. Handle 211 may also include a coupling 234 for attachment of a power source and a video monitor. Central portion 212 is coupled to handle 211, and is configured to be inserted into a puncture and navigate inside the body. Central portion 212 may be soft and flexible, or more rigid depending on the application and user preferences. Central portion 212 and/or deflectable portion 213 may have a cross-section that has a keyhole shape or any other suitable shape.

Deflectable portion 213 is coupled to central portion 212 and is also configured to be inserted into a puncture and navigate inside the body. Deflectable portion 213 may be deflected in one or more axes, as shown for example in FIG. 3B. Deflectable portion 213 may be controlled with pullwires 267 coupled to steering control 216. Deflectable portion 213 may be locked in a desired configuration using tension lock 217. Deflectable portion 213 includes a visualization element 240 and one or more lights 241.

Visualization element 240 and lights 241 may be coupled to a distal end 233 of deflectable portion 213. Alternatively, visualization element 240 and lights 241 may be coupled to the side or any other suitable location of deflectable portion 213. Visualization element 240 and lights 241 are coupled to visualization wires 265 and light wires 266 which pass through visualization lumen 261 to coupling 234. Visualization element 240 and lights 241 are covered by a lens 242. Lens 142 may have a hydrophobic coating or other coating to reduce adhesion of natural and synthetic materials that would obscure the image. Visualization element 240 may be turned on or off, or capture turned on or off using visualization control 218. Lights 241 may be turned on or off, or their intensity adjusted using light control 219.

Access element 250 is at least partially positioned within access lumen 215, and can slide and rotate within access lumen 215. Access element 250 may be used for injection of a liquid, passing of a guidewire 205, application of a vacuum, or any other suitable purpose. Access element 250 includes a proximal portion 251 and a distal portion 253. Proximal portion 251 may include a coupling 254. Distal portion 253 has a tip 255 that may be a blunt tip trocar, a blunt tip obturator, a sharp edge trocar, a sharp edge needle (e.g., Tuohy, epidural, biopsy), a guidewire tip, or any other suitable instrument. Access element 250 may be configured to work with an RF, microwave, cryoablation, high intensity focused ultrasound (HIFU), laser, or any other suitable energy source. Distal portion 253 may have depth markings. Distal portion 253 may be connected to an ohmmeter to measure impedance as the needle penetrates the pericardial membrane into the pericardial space. The impedance measurement may be used to provide an indication as to whether the pericardial membrane has been penetrated. Access element 250 may be moved and rotated by manipulating proximal portion 251.

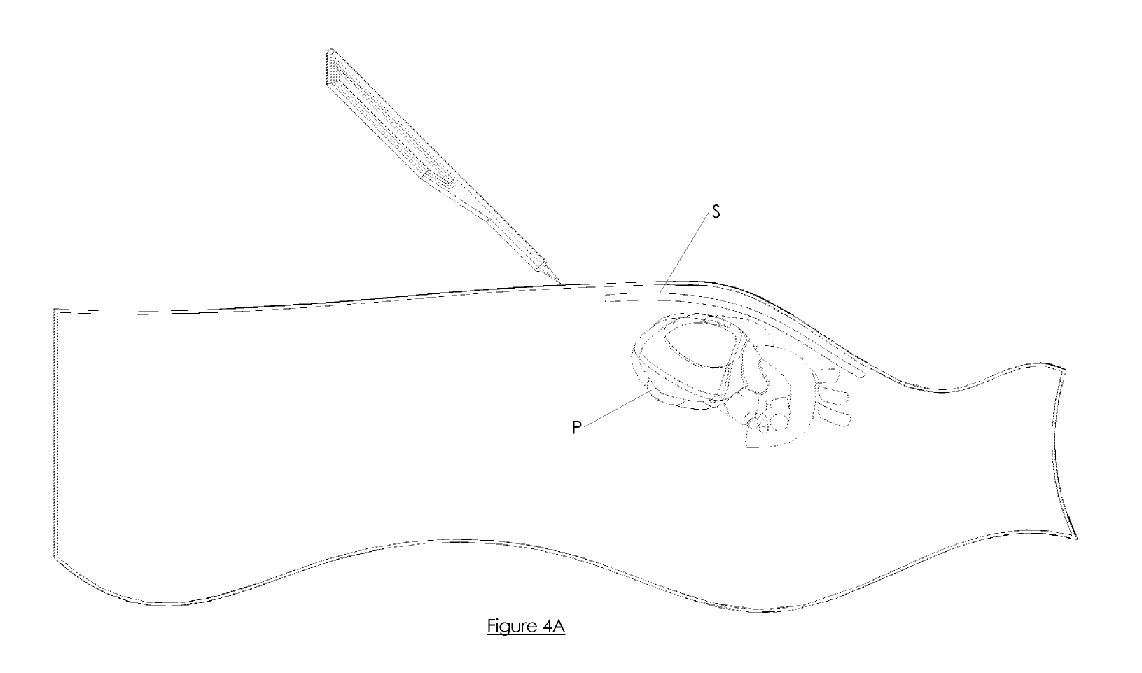

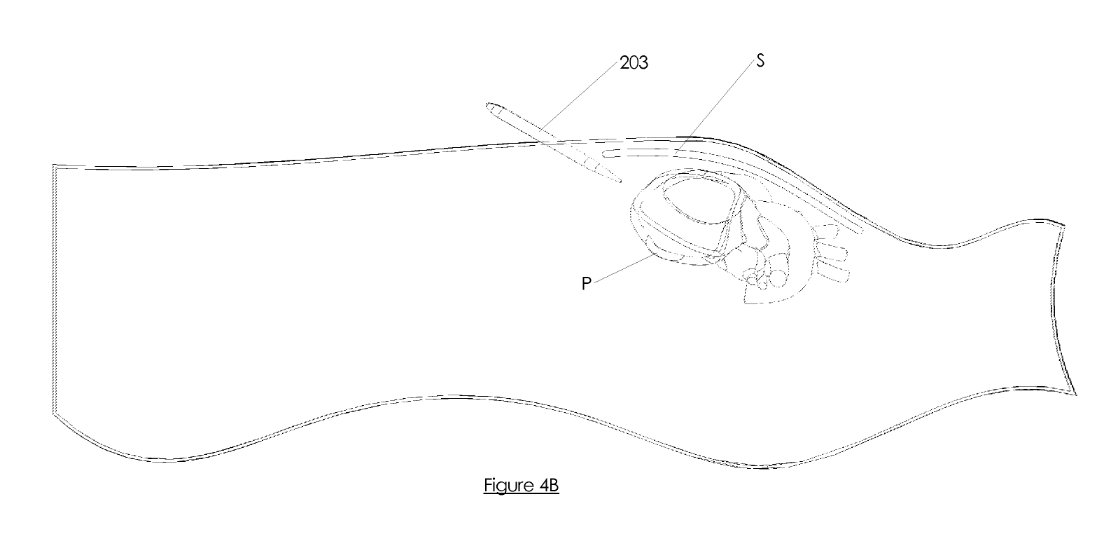

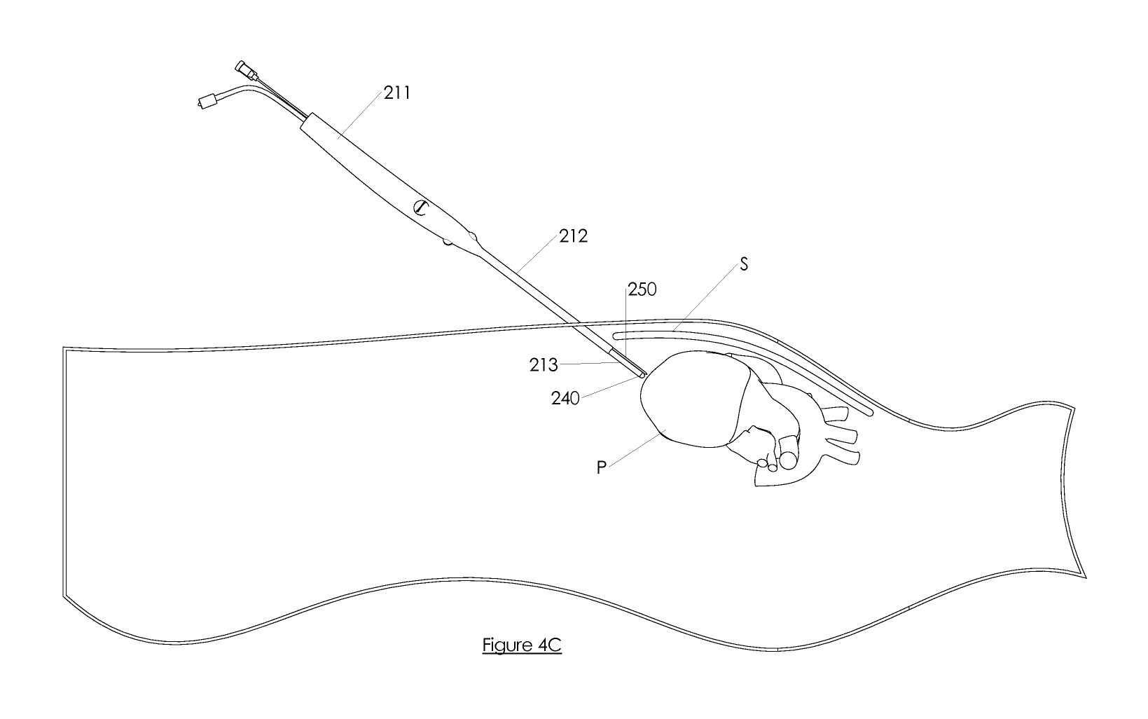

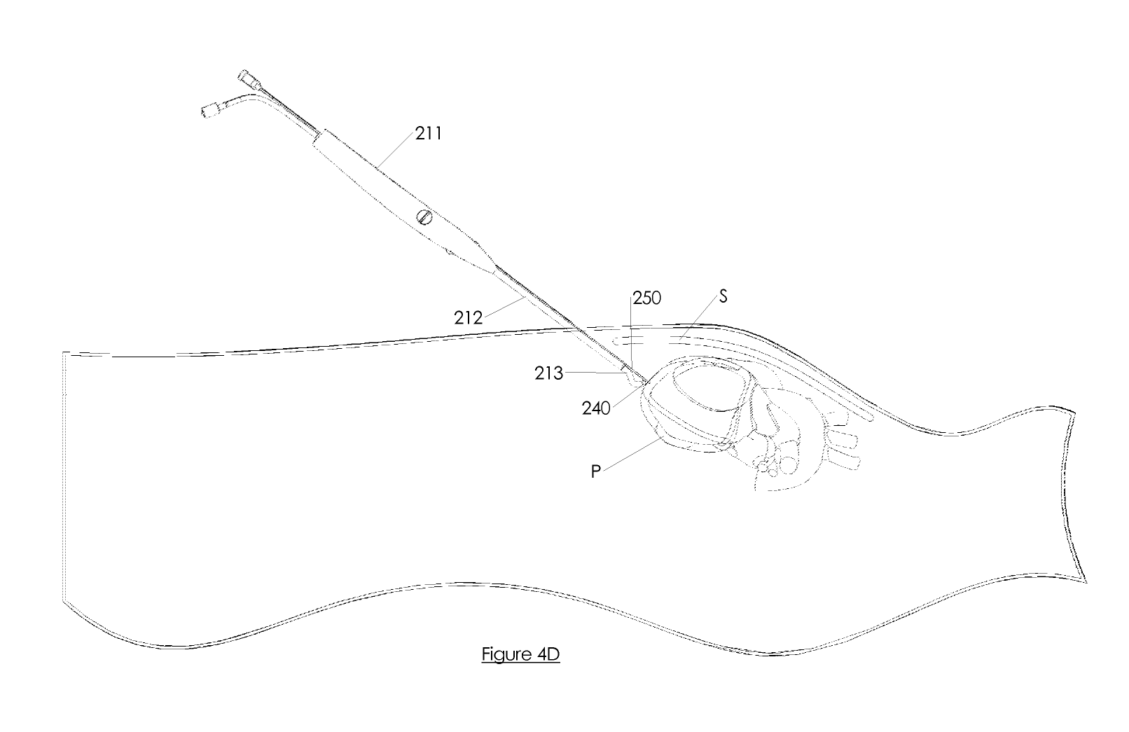

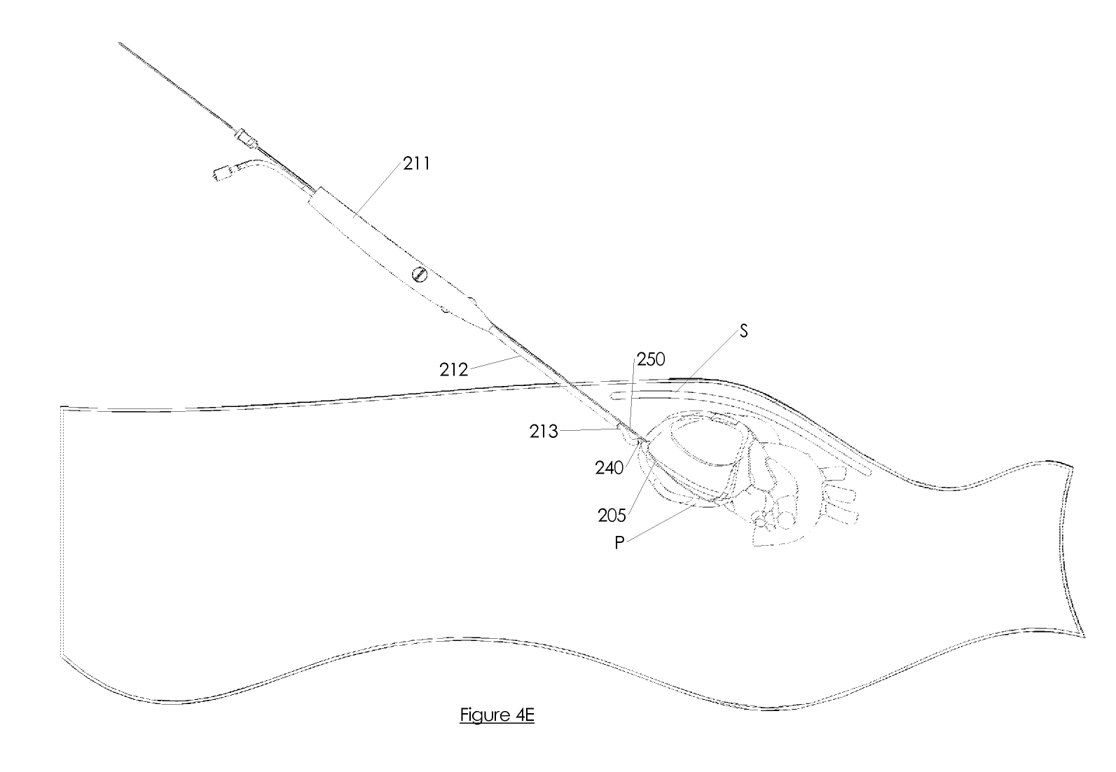

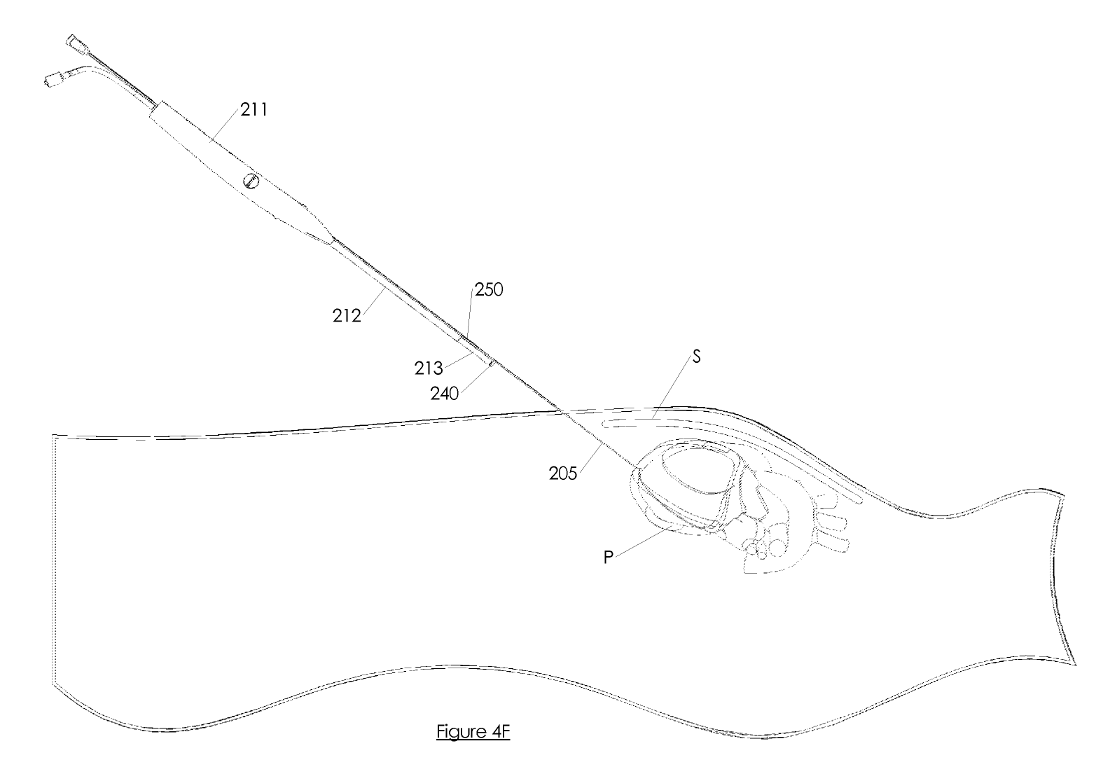

FIGS. 4A-4G show one method of using access device 200.

FIG. 4A shows a percutaneous puncture being made for a subxiphoid approach. Alternatively, an intercostal, apical, subclavian, suprasternal, or any other suitable approach may be used.

FIG. 4B shows a dilator 203 inserted through the puncture. Dilator 203 is used to dilate the puncture and then withdrawn.

FIG. 4C shows central portion 212 and deflectable portion 213 inserted through the puncture and positioned at or near the surface of the pericardium P. Visualization element 240 is used to guide central portion 212 and deflectable portion 213 along the posterior aspect of the sternum S to the surface of the pericardium P. Deflectable portion 213 may be manipulated using steering control 216. Access element 250 is retracted within distal end 233 of deflectable portion 213.

FIG. 4D shows access element 250 extended from distal end 233 of deflectable portion 213, and advanced through the pericardium P to create an access site. For an access element 250 having a sharp tip 255, visualization element 240 is used to visualize access element 250 as it is advanced through pericardium P. For an access element 250 used with RF energy, visualization element 240 is used to visualize access element 250 as RF energy is passed through access element 250 to penetrate pericardium P. Access element 250 may be rotated so that a desired surface is visible to visualization element 240. Saline, contrast, medications, and/or other fluids may be introduced through access element 250 into the pericardial space.

FIG. 4E shows guidewire 205 passed through access element 250 and positioned in the pericardial space.

FIG. 4F shows access element 250 retracted back into distal end 233 of deflectable portion 213, and central portion 212 and deflectable portion 213 withdrawn, leaving guidewire 205 in place.

FIG. 4G shows a sheath 280 advanced over guidewire 205 through the puncture and the access site and into the pericardial space. Other devices or guidewires may be advanced through sheath 280 to access the pericardial space. Saline, contrast, medications, and/or other fluids may be introduced through sheath 280 into the pericardial space.

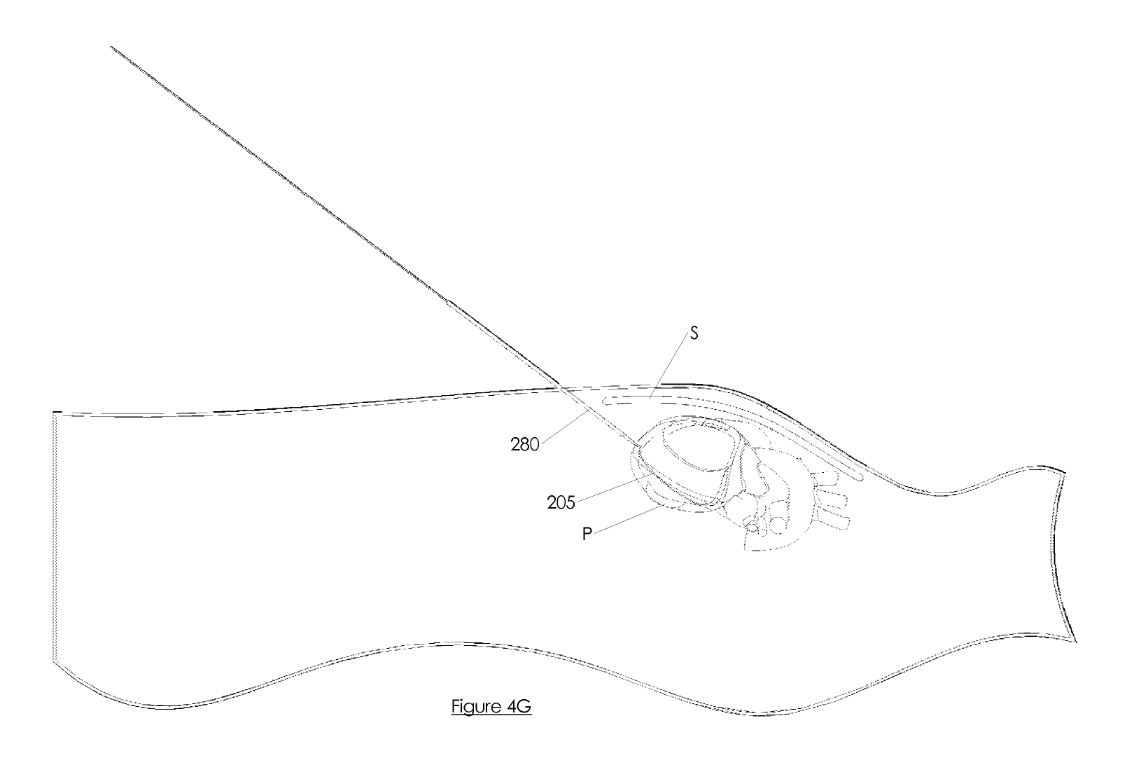

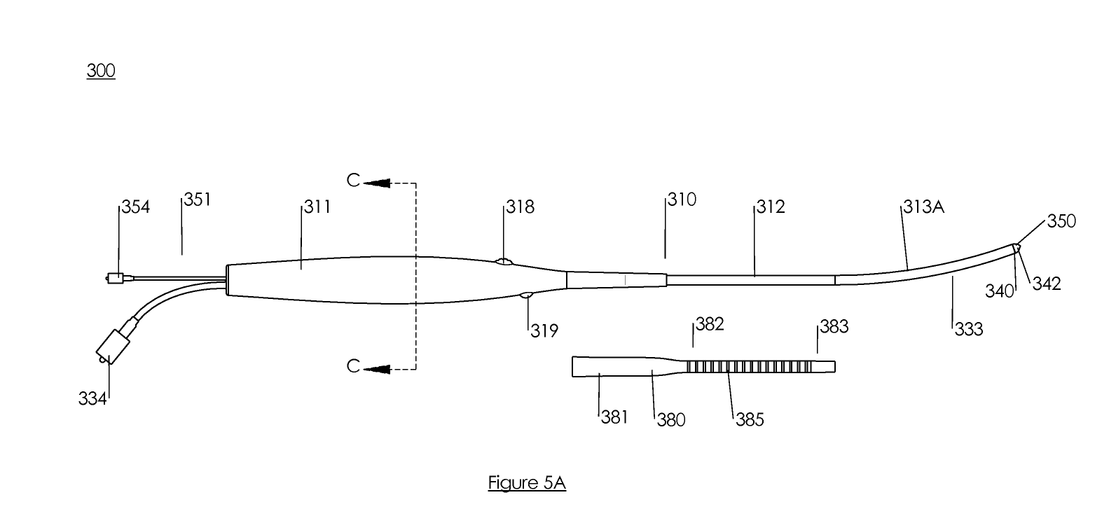

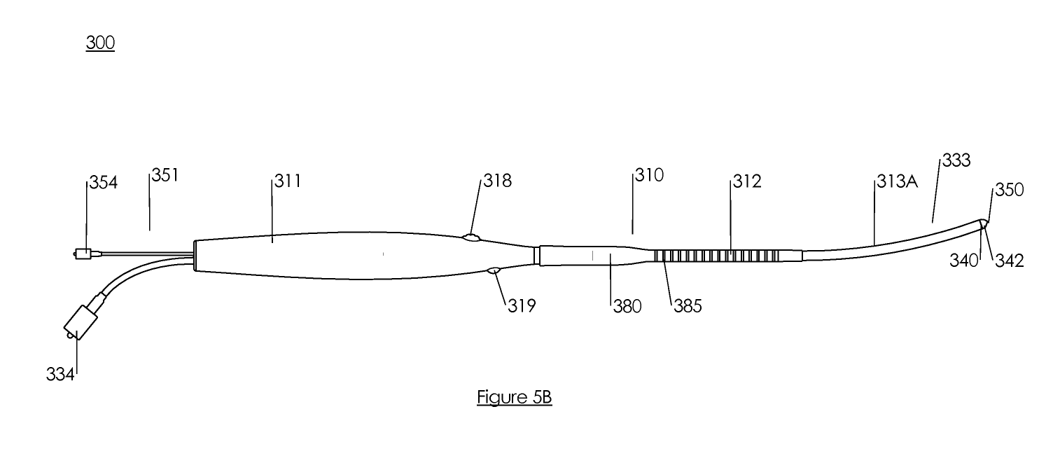

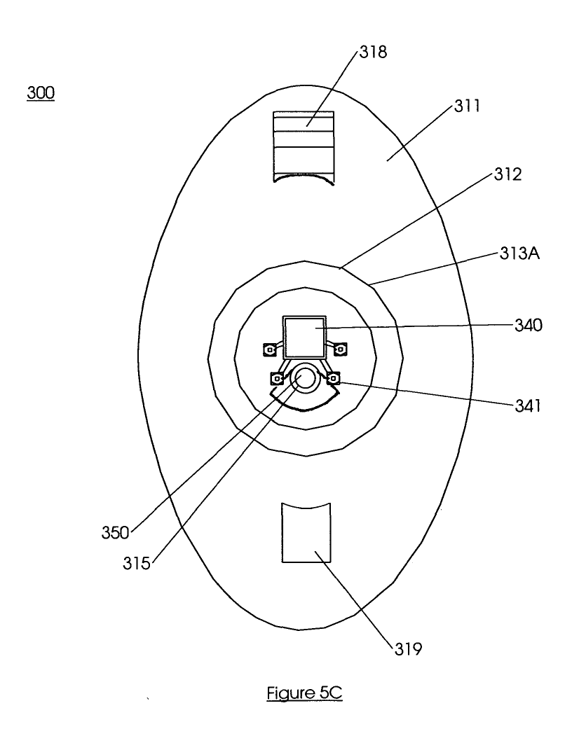

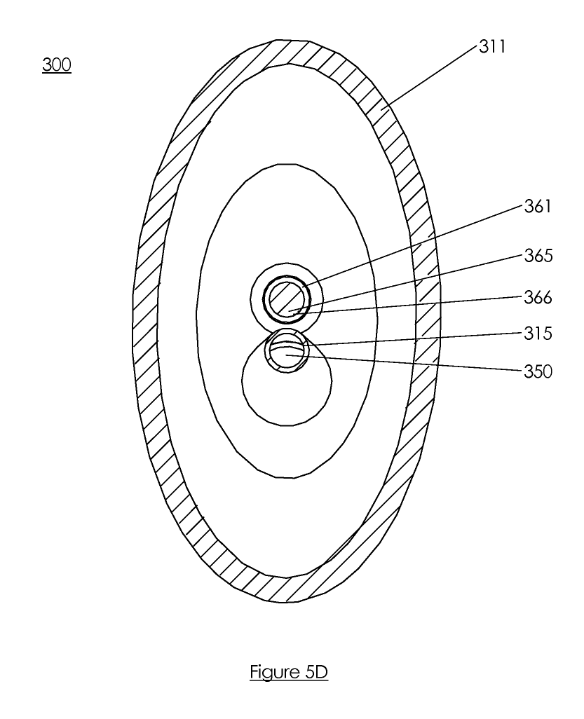

FIGS. 5A-5B show side views of yet another embodiment of an access device 300. FIG. 5C shows an end view of access device 300. FIG. 5D shows a cross-sectional end view of access device 300.

Access device 300 includes a housing 310, a visualization element 340, and an access element 350. Access device 300 may also include a sheath 380.

Housing 310 includes a handle 311, a central portion 312, and a distal portion 313A. Housing 310 also includes an access lumen 315 and a visualization lumen 361. Handle 311 includes a visualization control 318 and a light control 319. Handle 311 may also include a coupling 334 for attachment of a power source and a video monitor. Central portion 312 is coupled to handle 311, and is configured to be inserted into a puncture and navigate inside the body. Central portion 312 may be substantially rigid.

Distal portion 313A is coupled to central portion 312 and is also configured to be inserted into a puncture and navigate inside the body. Distal portion 313A may also be substantially rigid. Distal portion 313A may have a curved configuration, and may be bent or otherwise configured by the user and hold its shape. Distal portion 313A may include tubing made of a ductile material such as stainless steel or any other suitable material. Distal portion 313A includes a visualization element 340 and one or more lights 341.

Visualization element 340 and lights 341 may be coupled to a distal end 333 of distal portion 313A. Alternatively, visualization element 340 and lights 341 may be coupled to the side or any other suitable location of distal portion 313A. Visualization element 340 and lights 341 are coupled to visualization wires 365 and light wires 366 which pass through visualization lumen 361 to coupling 334. Visualization element 340, lights 341, and access lumen 315 are covered by a lens 342. Lens 342 includes an opening 343 which is continuous with access lumen 315. Lens 342 may also include a nozzle or other opening configured to clean lens 342. Lens 342 may have a hydrophobic coating or other coating to reduce adhesion of natural and synthetic materials that would obscure the image. Visualization element 340 may be turned on or off, or capture pictures or video using visualization control 318. Lights 341 may be turned on or off, or their intensity adjusted using light control 319.

Access element 350 is at least partially positioned within access lumen 315, and can slide and rotate within access lumen 315. Access element 350 may be used for injection of a liquid, passing of a guidewire 305, application of a vacuum, or any other suitable purpose. Access element 350 includes a proximal portion 351, a central portion 352, and a distal portion 353. Proximal portion 351 may include a coupling 354. Distal portion 353 has a tip 355 that may be a blunt tip trocar, a blunt tip obturator, a sharp edge trocar, a sharp edge needle (e.g., Tuohy, epidural, biopsy), a guidewire tip, or any other suitable instrument. Access element 350 may be configured to work with an RF, microwave, cryoablation, high intensity focused ultrasound (HIFU), laser, or any other suitable energy source. Distal portion 353 may have depth markings. Distal portion 353 may be connected to an ohmmeter to measure impedance as the needle penetrates the pericardial membrane into the pericardial space. The impedance measurement may be used to provide an indication as to whether the pericardial membrane has been penetrated. Access element 350 may be moved and rotated by manipulating proximal portion 351.

Central portion 352 is flexible, and capable of translating motions from proximal portion 351 to distal portion 353. Flexible central portion 352 allows access element 350 to move with distal portion 313A of housing 310. Central portion 352 may be constructed of a flexible braided material, a ductile metal, or any other suitable material. Proximal portion 351 may be substantially rigid. Distal portion 353 may be substantially rigid to facilitate penetration of tissue. Proximal portion 351, central portion 352, and distal portion 353 may be coupled with any suitable coupling device or method.

Sheath 380 includes a proximal portion 381, a central portion 382, and a distal portion 383. Proximal portion 381 may be grasped, and may include a coupling for attachment to an RF or other suitable energy source. Distal portion 383 may be made of a soft, flexible material and may stretch to fit snugly around housing 310. Central portion 382 may include electrodes 385 for coagulation and other purposes. Central portion 382 may have electrodes 385 that are configured circumferentially. Alternatively, electrodes 385 may be configured in a spiral, double helix, opposing helix, or any other suitable configuration. Electrodes 385 may be embedded in central portion 382 or otherwise coupled to central portion 382 in any suitable manner.

Sheath 380 may have a distal portion 383 that is tapered, with smaller end that tapers up in size towards central portion 382. The smaller end facilitates insertion of distal portion 383 into a puncture. The taper allows distal portion 383 to dilate the puncture as it is advanced. Electrodes 385 are configured to control bleeding proximate to the sheath at the site of the puncture, pericardium, or other structures.

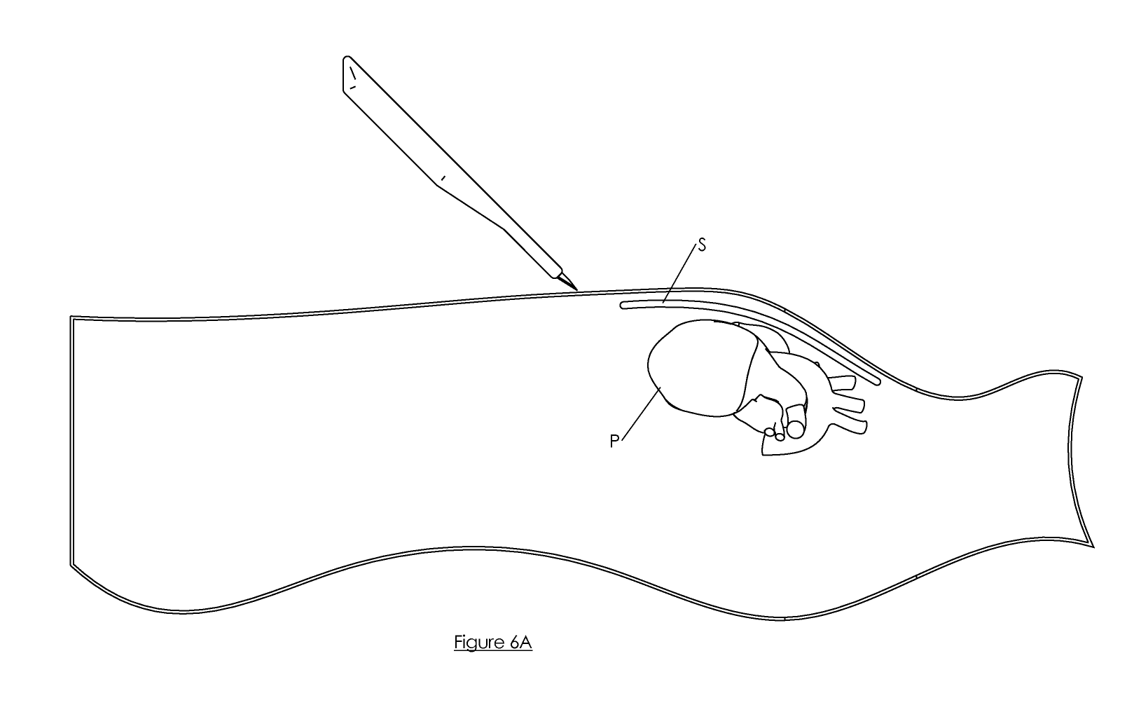

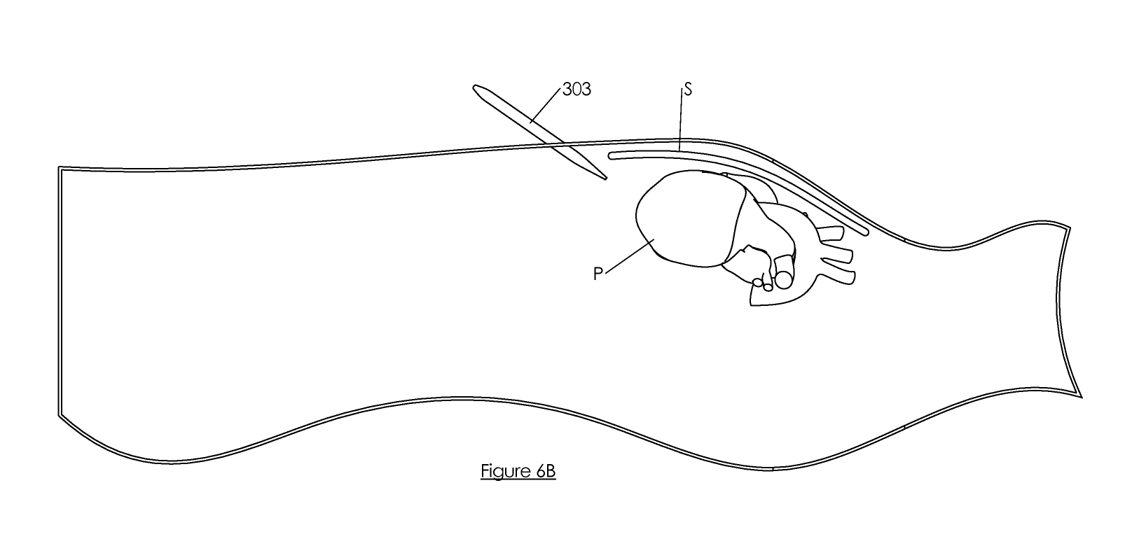

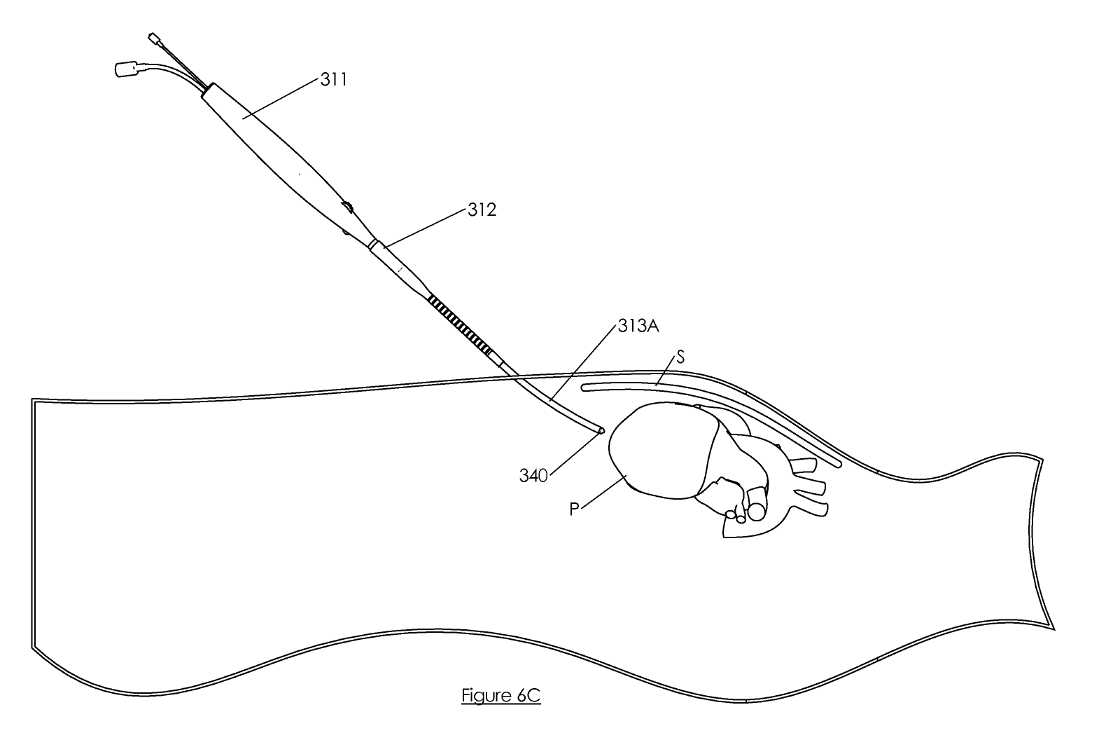

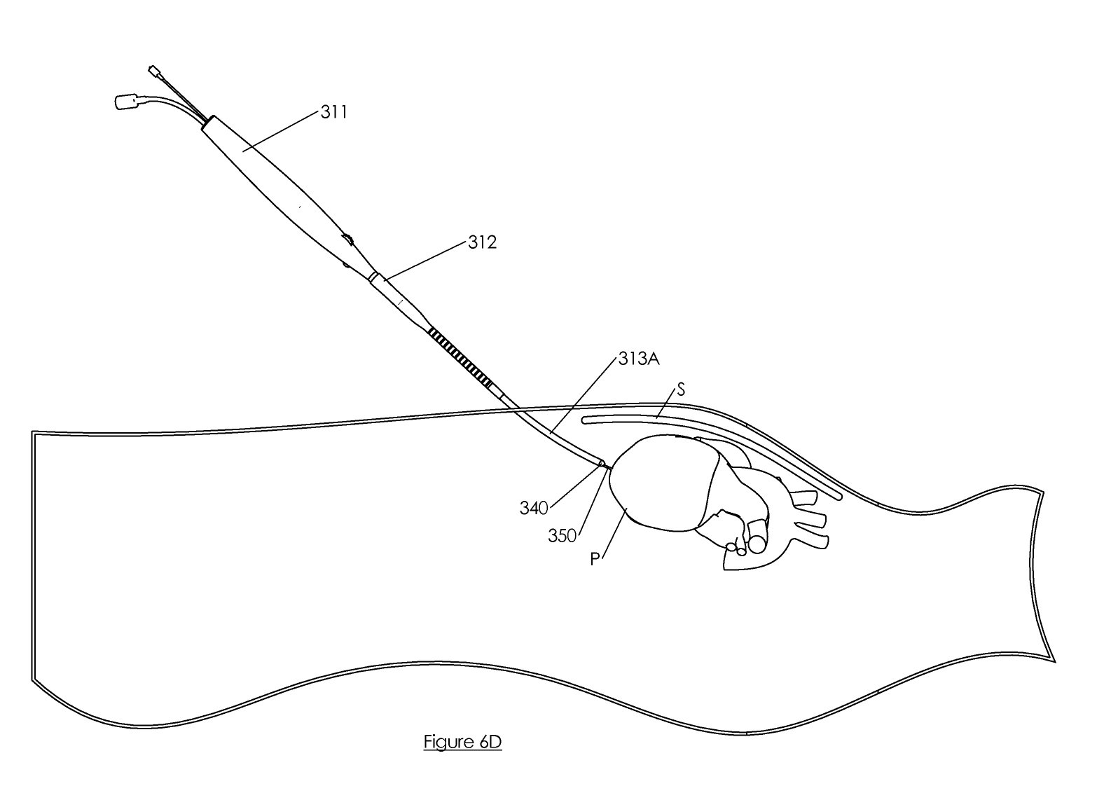

FIGS. 6A-6G show one method of using access device 300.

FIG. 6A shows a percutaneous puncture being made for a subxiphoid approach. Alternatively, an intercostal, apical, subclavian, suprasternal, or any other suitable approach may be used.

FIG. 6B shows a dilator 303 inserted through the puncture. Dilator 303 is used to dilate the puncture and then withdrawn.

FIG. 6C shows central portion 312 and distal portion 313A inserted through the puncture and positioned at or near the surface of the pericardium P. Visualization element 340 is used to guide central portion 312 and distal portion 313A along the posterior aspect of the sternum S to the surface of the pericardium P. Access element 350 is retracted within distal end 333 of distal portion 313A.

FIG. 6D shows access element 350 extended from distal end 333 of distal portion 313A, and advanced through the pericardium P to create an access site. For an access element 350 having a needle tip 355, visualization element 340 is used to visualize access element 350 as it is advanced through pericardium P. For an access element 350 used with RF energy, visualization element 340 is used to visualize access element 350 as RF energy is passed through access element 350 to penetrate pericardium P. Access element 350 may be rotated so that a desired surface is visible to visualization element 340. Saline, contrast, medications, and/or other fluids may be introduced through access element 350 into the pericardial space.

FIG. 6E shows guidewire 305 advanced through access element 350 and positioned in the pericardial space.

FIG. 6F shows access element 350 retracted back into distal end 333 of distal portion 313A, and central portion 312 and distal portion 313A withdrawn, leaving guidewire 305 in place.

FIG. 6G shows sheath 380 advanced over guidewire 305 through the puncture and the access site and into the pericardial space. Electrodes 385 may be used for coagulation. Other devices or guidewires may be advanced through sheath 380 to access the pericardial space. Saline, contrast, medications, and/or other fluids may be introduced through sheath 380 into the pericardial space.

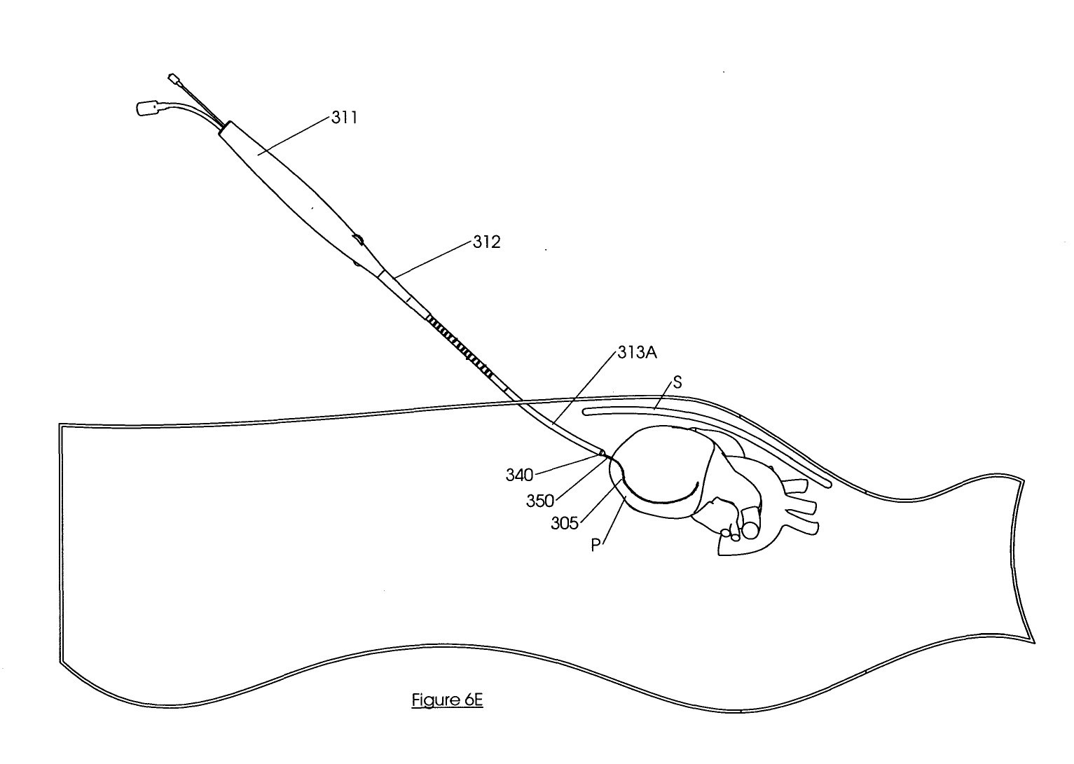

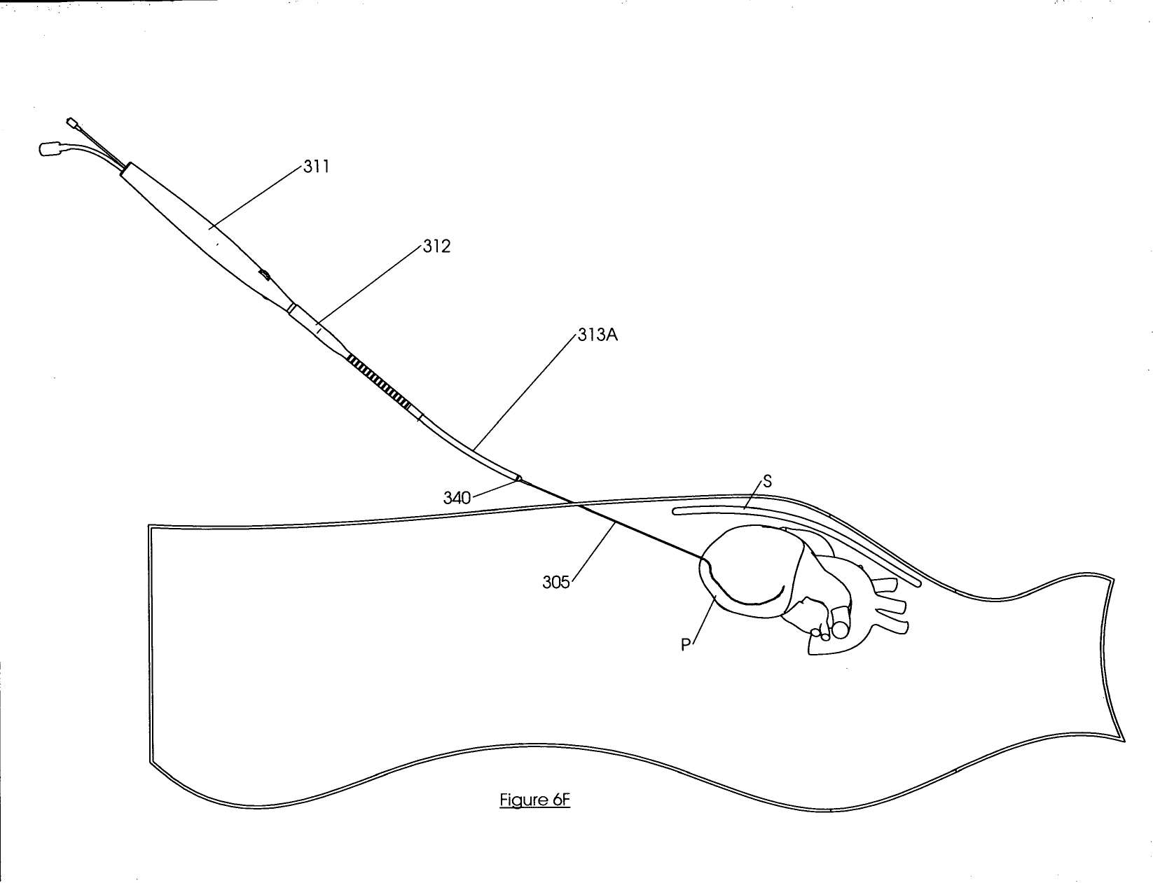

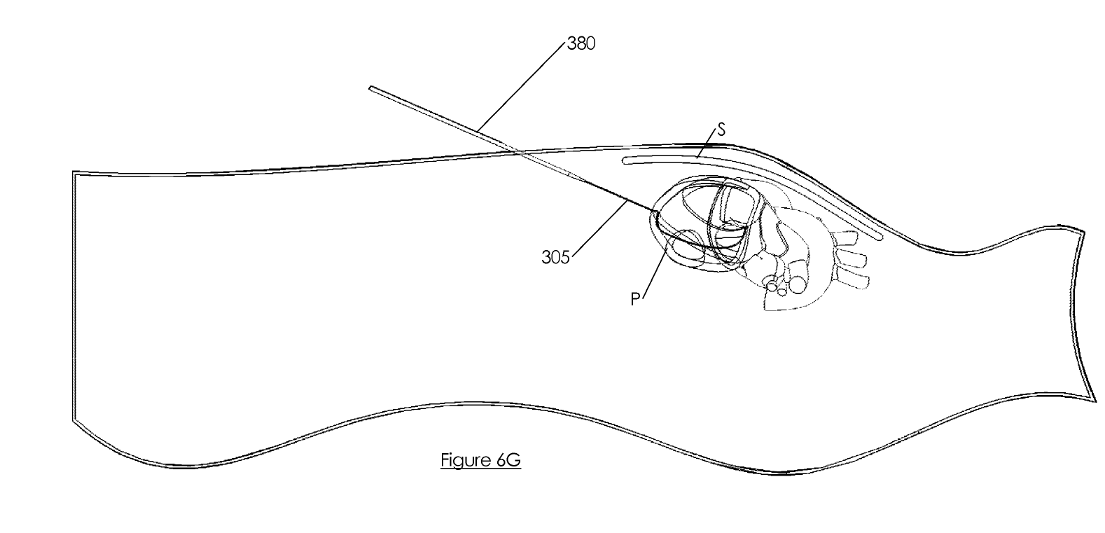

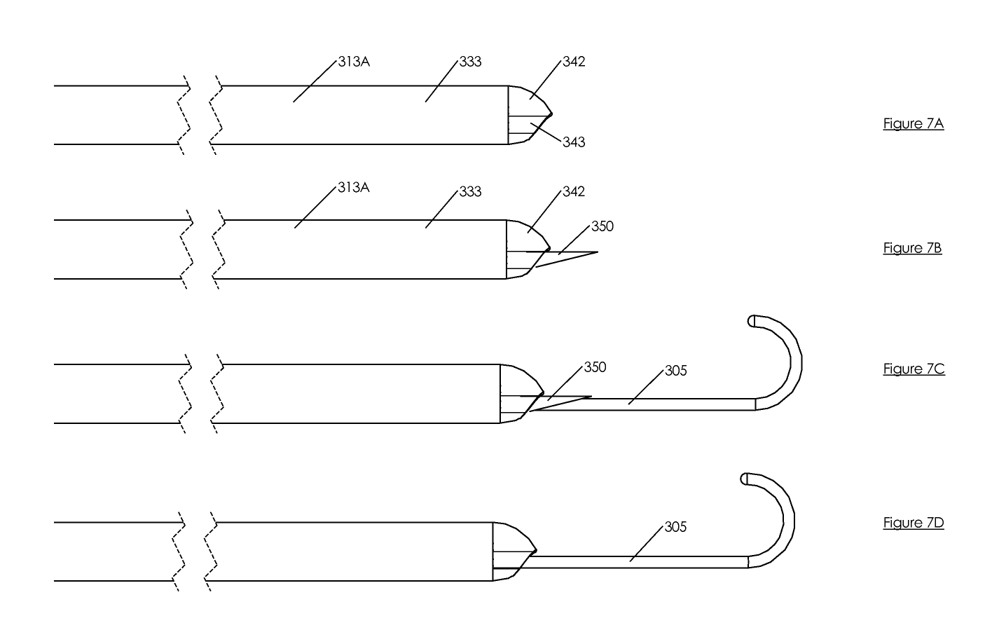

FIGS. 7A-7D show enlarged cross-sectional side views of distal portion 313A. FIG. 7A shows distal portion 313A with tip 355 of access element 350 retracted inside distal end 333. FIG. 7B shows distal portion 313A with tip 355 of access element 350 extended from distal end 333. FIG. 7C shows guidewire 305 advanced through access element 350. FIG. 7D shows tip of 355 of access element 350 pulled back inside distal end 333. Guidewire 305 remains in place.

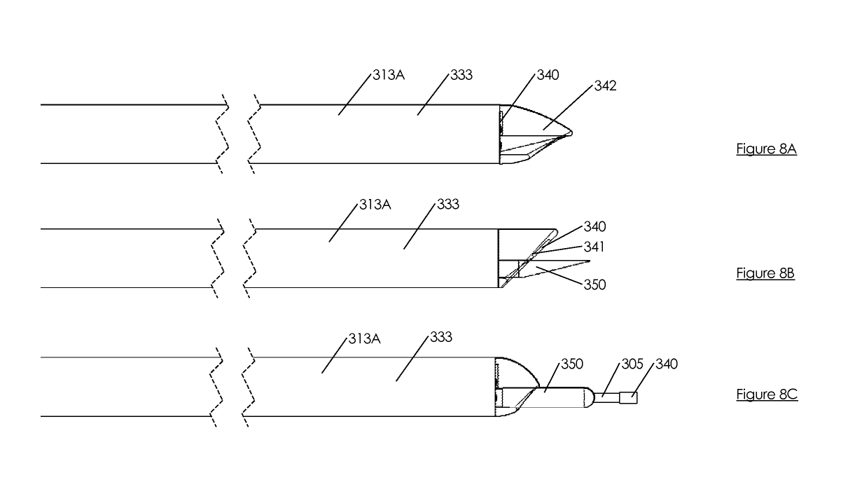

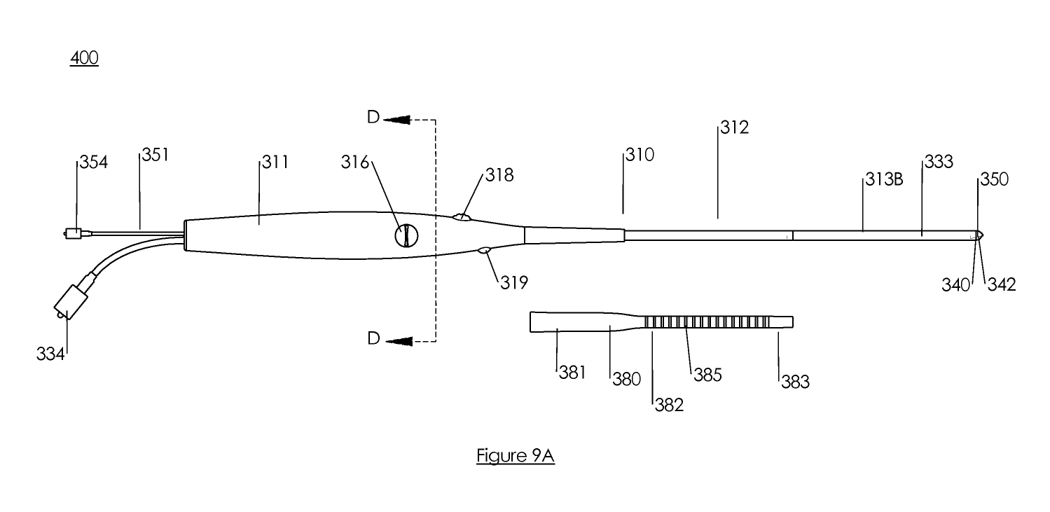

FIGS. 8A-8C show other embodiments of distal end 333 of distal portion 313A. FIG. 8A shows another embodiment of distal end 333 with lens 342 having a tapered profile. The tapered profile of distal end 333 may facilitate its advancement into the pericardial space. FIG. 8B shows yet another embodiment of distal end 333 having an asymmetrical tapered profile. Visualization element 340 and lights 341 may be mounted on the underside of the taper facing access element 350. The tapered profile of distal end 333 may facilitate its advancement into the pericardial space. FIG. 8C shows still another embodiment of distal end 333 with a visualization element 340 mounted on guidewire 305 and positioned within access element 350. Visualization element 340 is capable of being moved independently of access element 350.

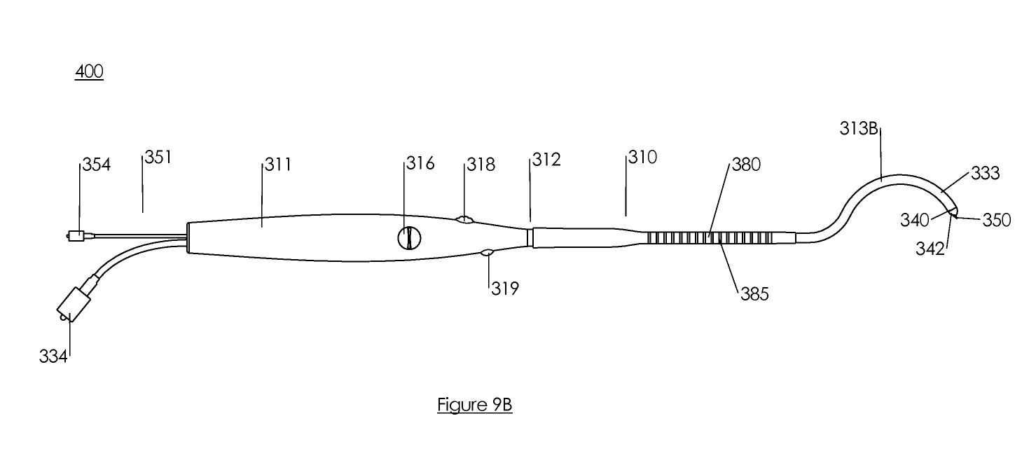

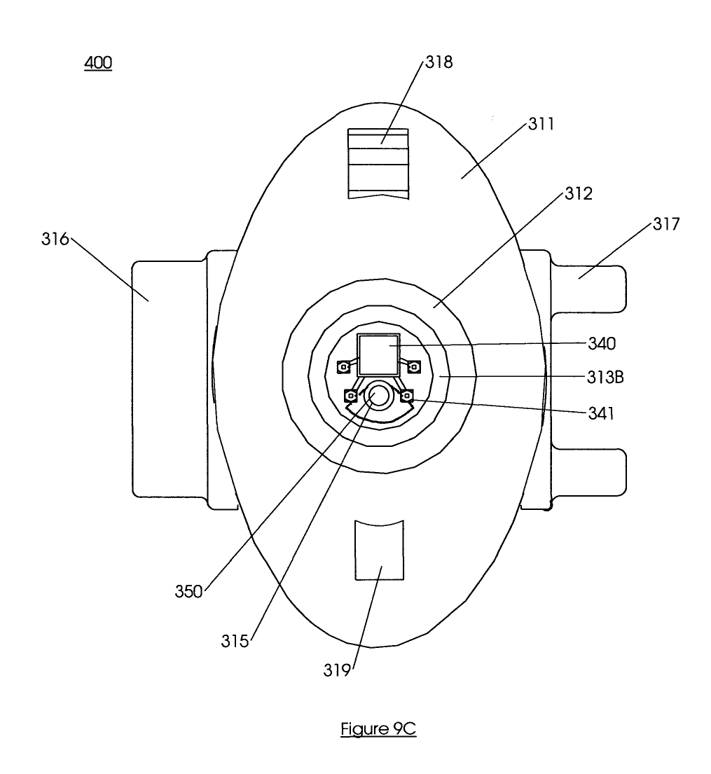

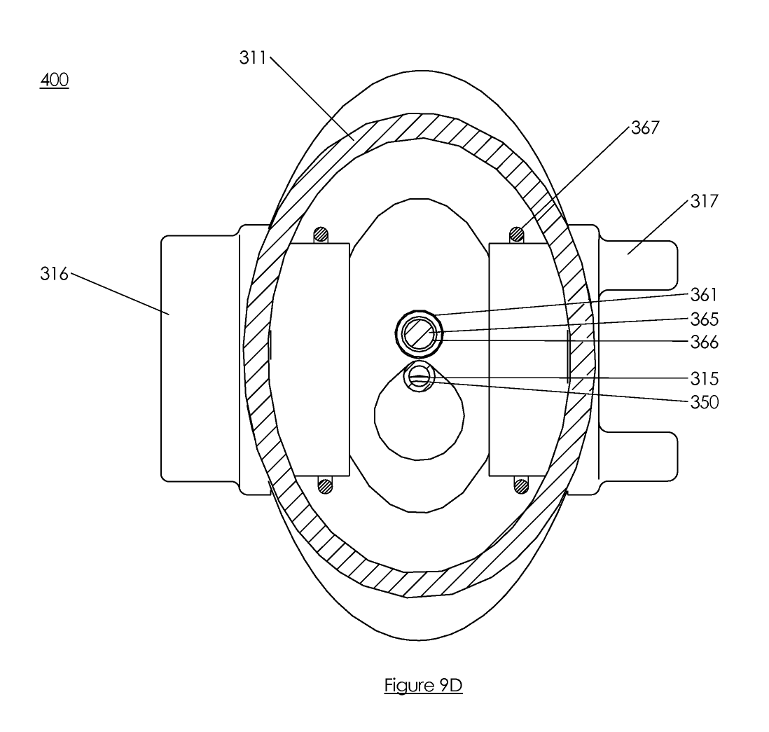

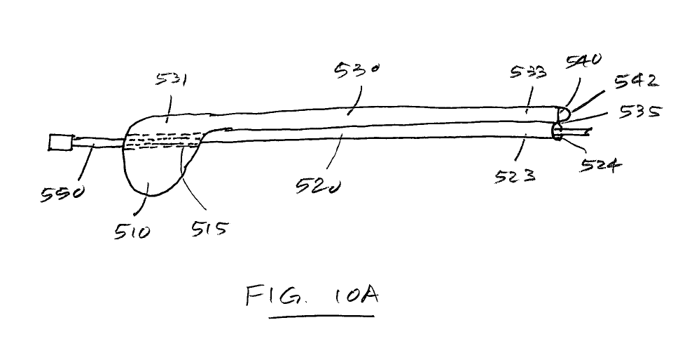

FIGS. 9A-9B show side views of still another embodiment of access device 400. FIG. 9C shows an end view of access device 400. FIG. 9D shows a cross-sectional end view of access device 400.

Access device 400 includes a housing 310, a visualization element 340, and an access element 350. Access device 400 may also include a sheath 380.

Housing 310 includes a handle 311, a central portion 312, and a deflectable portion 313B. Housing 310 also includes an access lumen 315 and a visualization lumen 361. Handle 311 includes a steering control 316, a tension lock 317, a visualization control 318, and a light control 319. Handle 211 may also include a coupling 334 for attachment of a power source and a video monitor. Central portion 312 is coupled to handle 311, and is configured to be inserted into a puncture and navigate inside the body. Central portion 312 may be soft and flexible, or more rigid depending on the application and user preferences.

Deflectable portion 313B is coupled to central portion 312 and is also configured to be inserted into a puncture and navigate inside the body. Deflectable portion 313B may be deflected in one or more axes, as shown for example in FIG. 5B. Deflectable portion 313B may be controlled with pullwires 367 coupled to steering control 316. Deflectable portion 313B may be locked in a desired configuration using tension lock 317. Deflectable portion 313B includes a visualization element 340 and one or more lights 341.

Access device 400 is similar to access device 300, but instead of a distal portion 313A that may be bent or otherwise configured by the user before being introduced into the body, access device 400 includes a deflectable portion 313B that is controlled by pullwires 367 coupled to steering control 316 and tension lock 317. The remainder of access device 400 is similar to access device 300. Access device 400 may be used in a manner similar to access device 300.

Access device 400 may have a central portion 312 that is lengthened. Access device 400 with a lengthened central portion 312 may be used to visualize and treat structures in the mediastinal space outside of the pericardium. Access device 400 with a lengthened central portion 312 may used to first create an entry site through the pericardium and introduce guidewire 305 into the pericardial space. Deflectable portion 313B may then be advanced over guidewire 305 through the entry site and into the pericardial space. Deflectable portion 313B may then be steered and navigated within the pericardial space to find a desired exit site. Deflectable portion 313B may then be used to create an exit site through the pericardium and access structures in the mediastinal space outside of the pericardium. Structures located posterior of the heart, superior to the diaphragm, and inferior to the clavicle such as the esophagus, trachea, primary bronchi, posterior pleural cavities, thoracic vertebrae and other structures may thus be accessed for delivery of therapeutics, biopsy, fixation, ablation, survey, and other purposes.

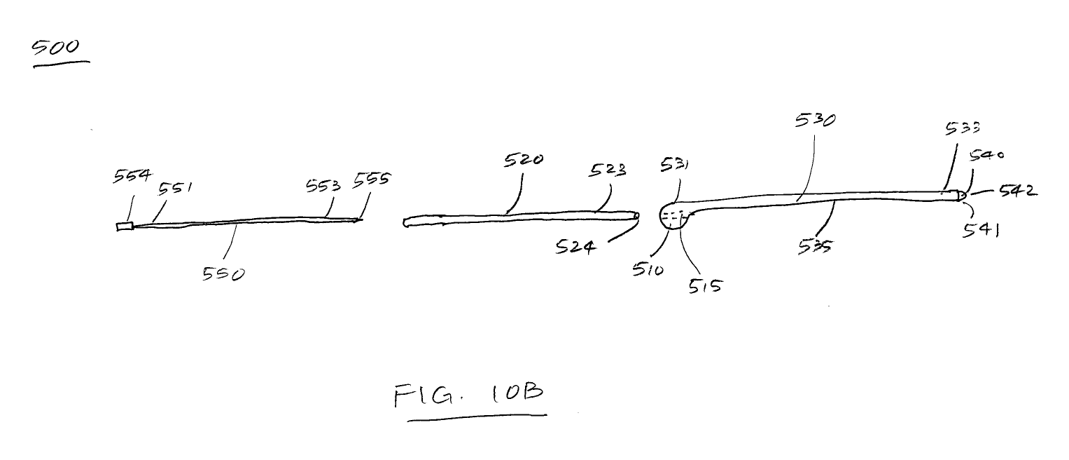

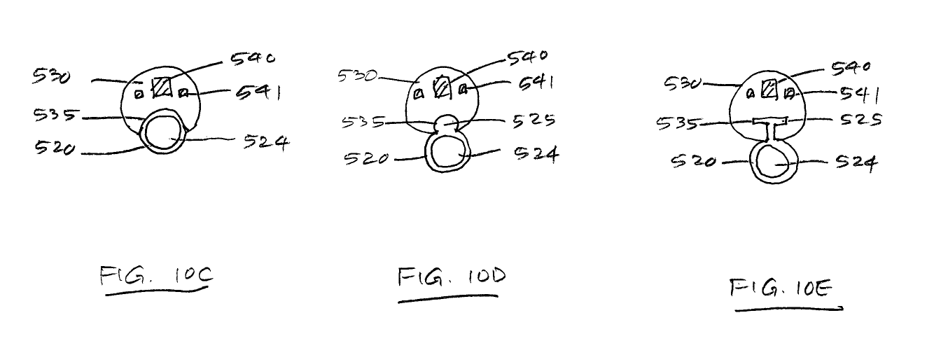

FIGS. 10A-10B show side and exploded views of one embodiment of an access device 500. FIG. 10C shows a cross-sectional view of access device 500. FIGS. 10D-10E show cross-sectional views of alternative embodiments of access device 500.

Access device 500 includes a visualization catheter 530 with a track 535, a working catheter 520, and a visualization element 540.

Visualization catheter 530 is configured to be inserted through a percutaneous puncture and navigate inside a body. Visualization catheter 530 may be rigid or flexible. Visualization catheter 530 may be straight or curved, or may be bent or otherwise configured by a user and hold its shape. Visualization catheter 530 includes a track 535 formed along its length. Track 535 is open to an exterior of visualization catheter 530.

Working catheter 520 is configured to slide along track 535. Working catheter 520 includes a working lumen 524 which allows a working element 550 to insert through. Working catheter 520 may be made of plastic, fabric, or any other suitable material.

As shown in FIG. 10C, working catheter 520 may have a cross section that is substantially circular, and track 535 may have a cross section that is substantially semicircular and receives working catheter 520. Alternatively, as shown in FIGS. 10D-10E, working catheter 520 may include a runner 525 that is configured to slide at least partially within track 535. Runner 525 may extend along the length of working catheter 520, or only along a portion of working catheter 520, such as at a distal end 523 of working catheter 520. Runner 525 and track 535 may be any suitable shape. FIG. 10D shows a working catheter 520 with a runner 525 that is substantially circular, and a track 535 that is also substantially circular. FIG. 10E shows a working catheter 520 with a runner 525 that is T-shaped, and a track 535 that is also T-shaped.

Track 535 may have a stop at a distal end 533 of visualization catheter 530 which prevents distal end 523 of working catheter 520 from traveling beyond distal end 533 of visualization catheter 530.

Visualization element 540 and one or more illumination elements 541 may be coupled to a distal end 533 of visualization catheter 530. Alternatively, visualization element 540 and illumination elements 541 may be coupled to the side or any other suitable location of visualization catheter 530, or mounted at a suitable angle to improve visualization. Visualization element 540 may include an imaging element with a pinhole aperture and/or one or more lenses. Visualization element 540 and illumination elements 541 may be covered by a cover 542. Cover 542 may include a nozzle or other opening configured to clean cover 542. Cover 542 may have a hydrophilic coating, a hydrophobic coating, or other coating to reduce adhesion of natural and synthetic materials that would obscure the image. Visualization element 540 may have a focal length selected for use with a typical working distance of working element 550, or be focused on an interior surface of cover 542.

A working element 550 may be inserted through working lumen 524 of working catheter 520, and can slide and rotate within working lumen 24. Working element 550 may be used for injection of a liquid, passing of a guidewire 505, application of a vacuum, or any other suitable purpose. Working element 550 includes a proximal portion 551, a central portion 552, and a distal portion 553. Proximal portion 551 may include a coupling 554. Distal portion 553 has a tip 555 that may be a blunt tip trocar, a blunt tip obturator, a sharp edge trocar, a sharp edge needle (e.g., Tuohy, epidural, biopsy), a guidewire tip, or any other suitable instrument. Working element 550 may be configured to work with an RF, microwave, cryoablation, high intensity focused ultrasound (HIFU), laser, or any other suitable energy source. Distal portion 553 may have depth markings. Distal portion 553 may be connected to an ohmmeter to measure impedance as the needle penetrates the pericardial membrane into the pericardial space. The impedance measurement may be used to provide an indication as to whether the pericardial membrane has been penetrated. Working element 550 may be moved and rotated by manipulating proximal portion 551.

Visualization catheter 530 may include at its proximal end 531 a handle 510. Handle 510 may include an opening 515 configured to receive working catheter 520. Opening 515 aligns working catheter 520 with track 535. Opening 515 may have a shape similar to a cross section of working catheter 520. If working catheter 520 includes a runner 525, opening 515 and may help “thread” runner 525 into track 535.

Working catheter 520 may be configured to fit loosely around working element 550, or to reduce the amount of friction or drag on working element 550. Working catheter 520 allows working element 550 to move freely within working lumen 524 and enhance the “feel” and control of position at a proximal portion 551 of working element 550 of what is being accessed at a distal portion 553 of working element 550. Working catheter 520 may have a length substantially similar to that of visualization catheter 530.

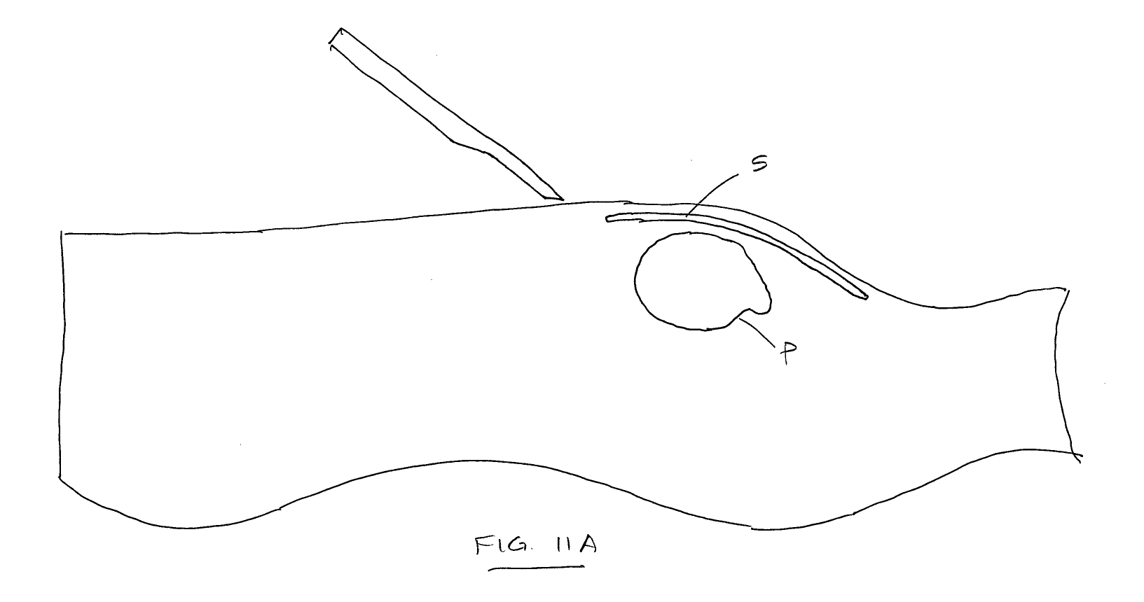

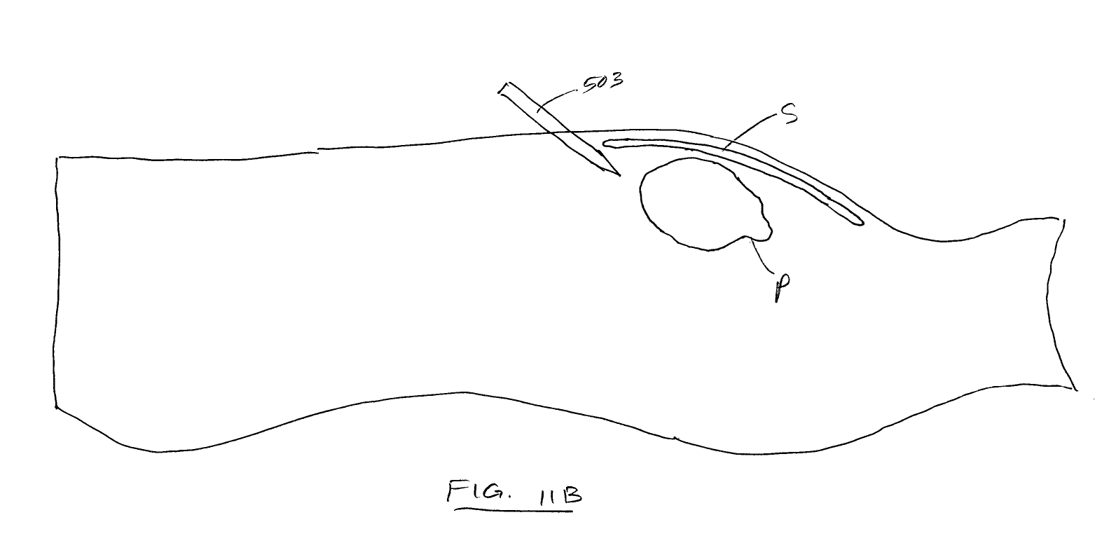

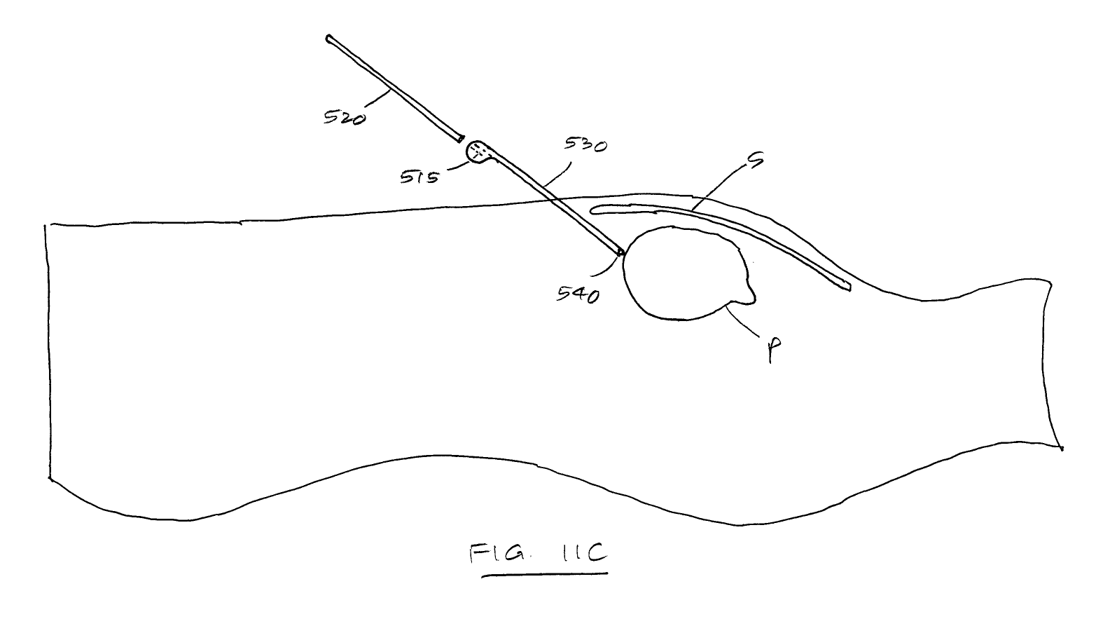

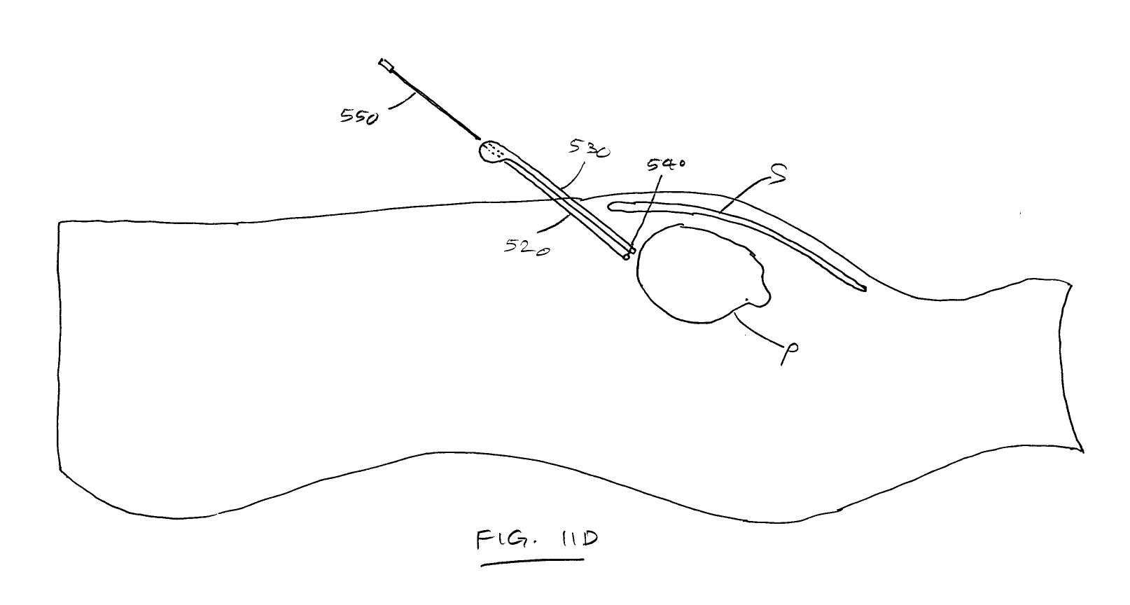

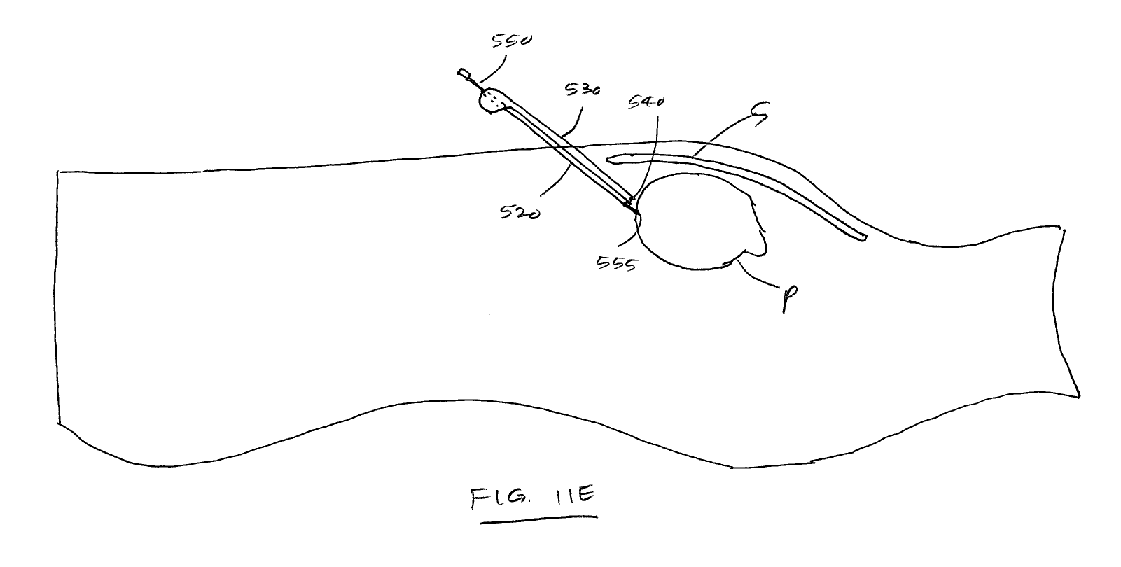

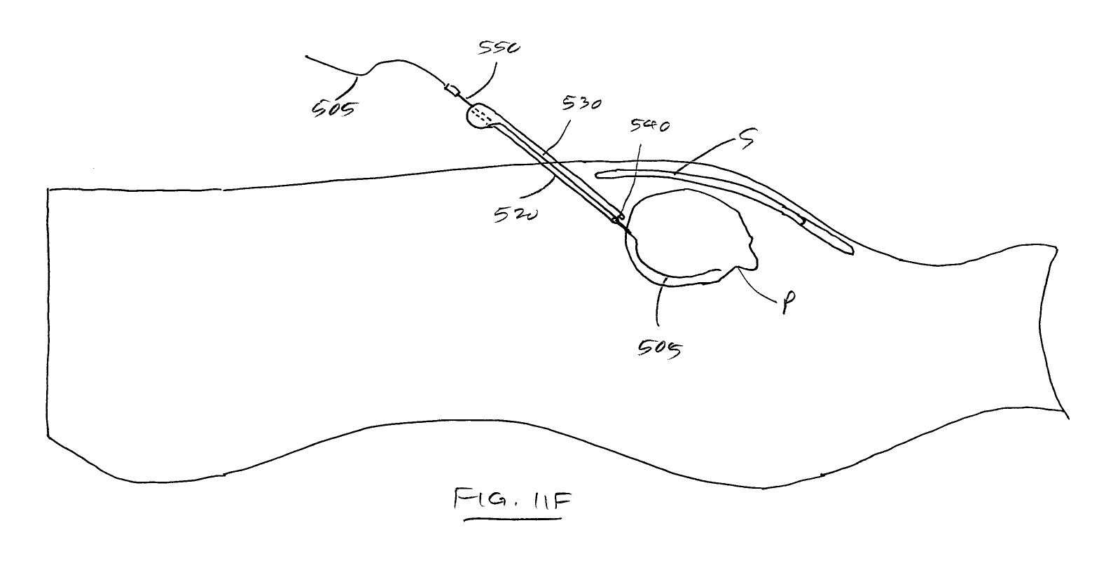

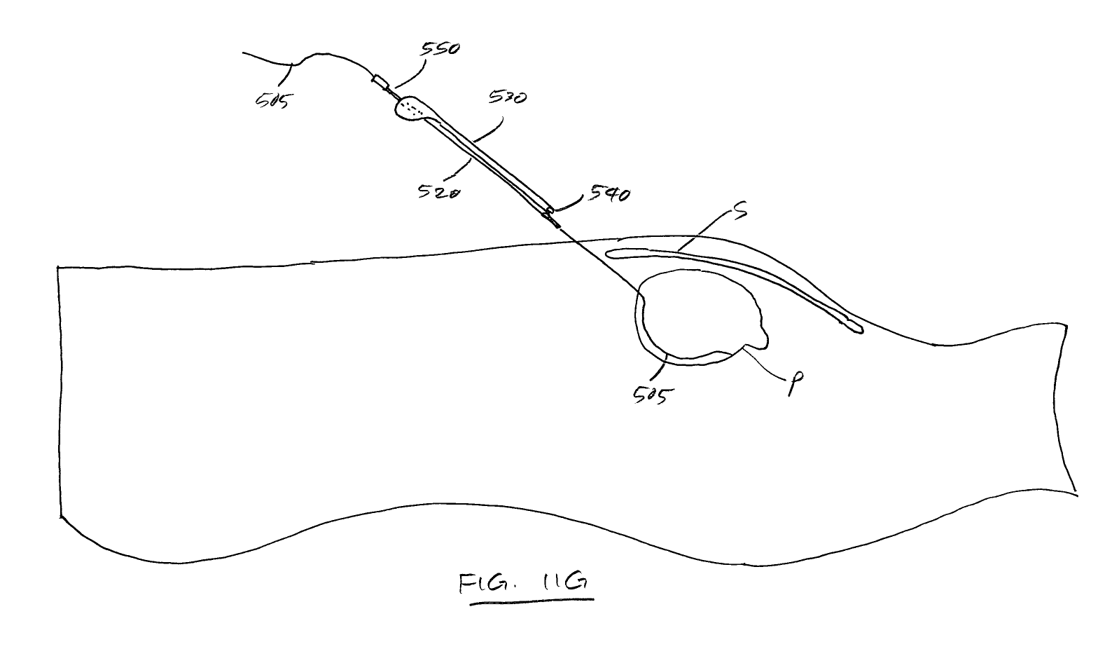

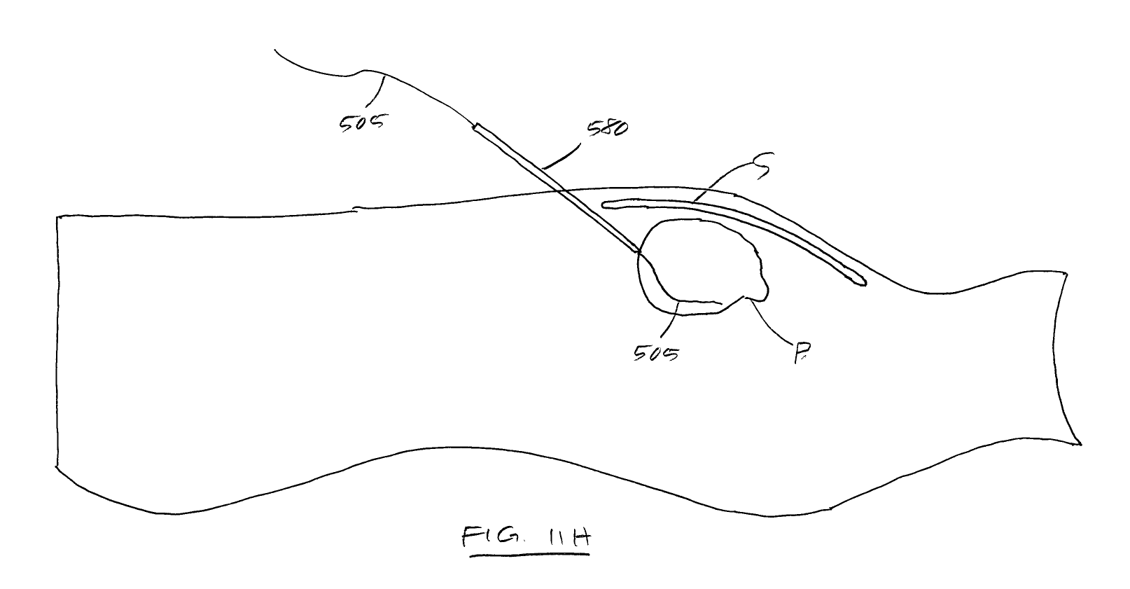

FIGS. 11A-11H show one method of using access device 500.

FIG. 11A shows a percutaneous puncture being made for a subxiphoid approach. Alternatively, an intercostal, apical, subclavian, suprasternal, or any other suitable approach may be used.

FIG. 11B shows a dilator 503 inserted through the puncture. Dilator 503 is used to dilate the puncture and then withdrawn.

FIG. 11C shows visualization catheter 530 inserted through the puncture and its distal end 533 positioned at or near the surface of the pericardium P. Visualization element 540 is used to guide visualization catheter 530 along the posterior aspect of the sternum S to the surface of the pericardium P.

FIG. 11D shows working catheter 520 inserted through opening 515 in handle 510 and along track 535 through the puncture, until distal end 523 of working catheter 520 is in the vicinity of visualization element 540.

FIG. 11E shows a working element 550 passed through working lumen 524 of working catheter 520, and advanced through the pericardium P to create an access site. For a working element 550 having a needle tip 555, visualization element 540 is used to visualize working element 550 as it is advanced through pericardium P. For a working element 550 used with RF energy, visualization element 540 is used to visualize working element 550 as RF energy is passed through working element 550 to penetrate pericardium P. Working element 550 may be rotated so that a desired surface is visible to visualization element 540. Saline, contrast, medications, and/or other fluids may be introduced through working element 550 into the pericardial space.

FIG. 11F shows guidewire 505 advanced through working element 550 and positioned in the pericardial space.

FIG. 11G shows visualization catheter 530, working catheter 520, and working element 550 withdrawn, leaving guidewire 505 in place.

FIG. 11H shows a sheath 580 advanced over guidewire 505 through the puncture and the access site and into the pericardial space. Other devices or guidewires may be advanced through sheath 580 to access the pericardial space. Saline, contrast, medications, and/or other fluids may be introduced through sheath 580 into the pericardial space.

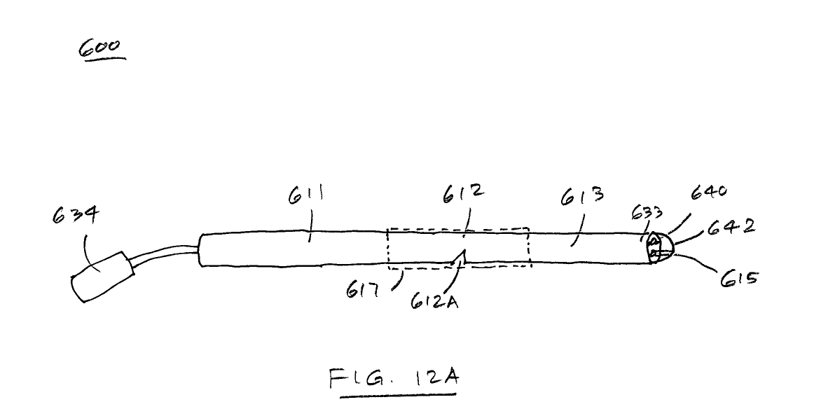

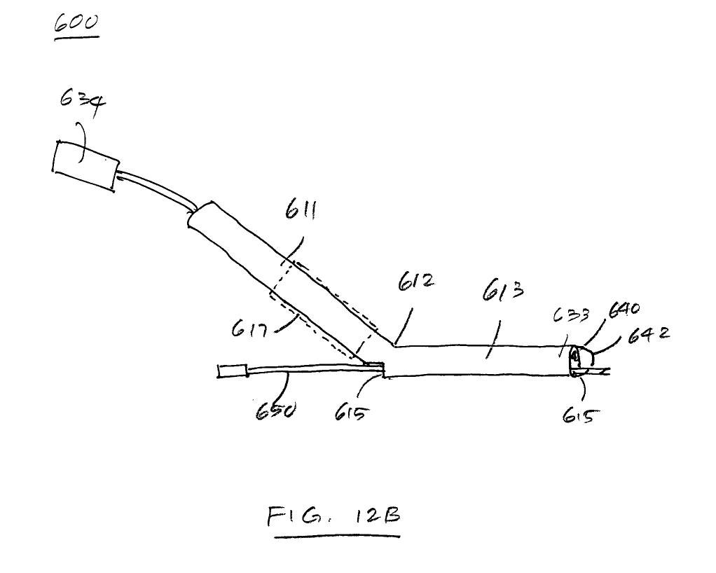

FIGS. 12A-12B show side views of another embodiment of an access device 600. FIG. 12C shows an cross-sectional view of access device 600 at a break 620. FIGS. 12D-12E show alternative embodiments of access device 600.

Access device 600 includes a catheter 610 having a proximal segment 611 and a distal segment 613 coupled by a joint 612, and a visualization element 640.

Catheter 610 includes a proximal segment 611 and a distal segment 613. A visualization lumen 661 extends continuously through proximal segment 611 and distal segment 613. A distal working lumen 615 extends through distal segment 613. Catheter 610 may include a coupling 634 for attachment of a power source, video monitor, and/or controls.

Proximal segment 611 and distal segment 613 are coupled by a joint 612. Joint 612 may be opened to provide access to distal working lumen 615 without breaking visualization lumen 661. Joint 612 may be a cut or notch 612A passing across distal working lumen 615 which does not impinge on visualization lumen 661. Catheter 610 may be made of a material that is sufficiently flexible to allow catheter 610 to flex or bend at joint 612 and provide access to distal working lumen 615. Catheter 610 may be able to bend as much as is necessary to allow access to distal working lumen 615. Alternatively, joint 612 may be a hole or aperture formed in a side of catheter 610 which may be bent to provide access to distal working lumen 615. A removable sleeve 617 may be used over joint 612 to prevent joint 612 from bending until needed. Alternatively, pullwires may be used to lock joint 612 in open and closed positions. Catheter 610 may include other lumens 662 such as irrigation and vacuum lumens which, like visualization lumen 661, are not broken by joint 612.

Alternatively, joint 612 may be a hinge 612B as shown in FIG. 12D which may be rotated to be opened, or a swivel 612C as shown in FIG. 12E which may be rotated to open joint 612 and provide access to distal working lumen 615. Catheter 610 with a hinge 612B or a swivel 612C may be made of a flexible or a rigid material.

Distal segment 613 is configured to be inserted through a percutaneous puncture and navigate inside the body. Distal segment 613 may have a curved configuration, and may be bent or otherwise configured by the user and hold its shape.

Visualization element 640 and one or more illumination elements 641 may be coupled to a distal end 633 of distal segment 613. Alternatively, visualization element 640 and illumination elements 641 may be coupled to the side or any other suitable location of distal segment 613, or mounted at a suitable angle to improve visualization. Visualization element 640 may include an imaging element with a pinhole aperture and/or one or more lenses. Visualization element 640, illumination elements 641, and distal working lumen 615 may be covered by a cover 642. Cover 642 may include a channel 643 which is continuous with distal working lumen 615. Alternatively, cover 642 may cover only visualization element 640 and illumination elements 641, and not distal working lumen 615. Cover 642 may include a nozzle or other opening configured to clean cover 642. Cover 642 may have a hydrophilic coating, a hydrophobic coating, or other coating to reduce adhesion of natural and synthetic materials that would obscure the image. Visualization element 640 may have a focal length selected for use with a typical working distance of working element 650, or be focused on an interior surface of cover 642.

A working element 650 may be inserted through distal working lumen 615, and can slide and rotate within distal working lumen 615. Working element 650 may be used for injection of a liquid, passing of a guidewire 605, application of a vacuum, or any other suitable purpose. Working element 650 includes a proximal portion 651, a central portion 652, and a distal portion 653. Proximal portion 651 may include a coupling 654. Distal portion 653 has a tip 655 that may be a blunt tip trocar, a blunt tip obturator, a sharp edge trocar, a sharp edge needle (e.g., Tuohy, epidural, biopsy), a guidewire tip, or any other suitable instrument. Working element 650 may be configured to work with an RF, microwave, cryoablation, high intensity focused ultrasound (HIFU), laser, or any other suitable energy source. Distal portion 653 may have depth markings. Distal portion 653 may be connected to an ohmmeter to measure impedance as the needle penetrates the pericardial membrane into the pericardial space. The impedance measurement may be used to provide an indication as to whether the pericardial membrane has been penetrated. Working element 650 may be moved and rotated by manipulating proximal portion 651.

Joint 612 reduces the length of distal working lumen 615. This enhances the “feel” and control of position at a proximal portion 651 of working element 650 of what is being accessed at a distal portion 653 of working element 650. Also, a shorter distal working lumen 615 may allow a greater range of off-the-shelf needles to be used as working element 650. A first user may manipulate working element 650, while a second user may operate catheter 610 and visualization element 640 and other functions such as vacuum and irrigation.

Alternatively, proximal segment 611 may include a proximal working lumen which aligns with distal working lumen 615 when joint 612 is closed, and catheter 610 may be used with a full-length working lumen.

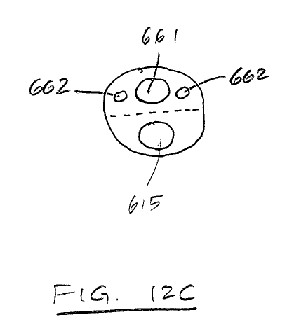

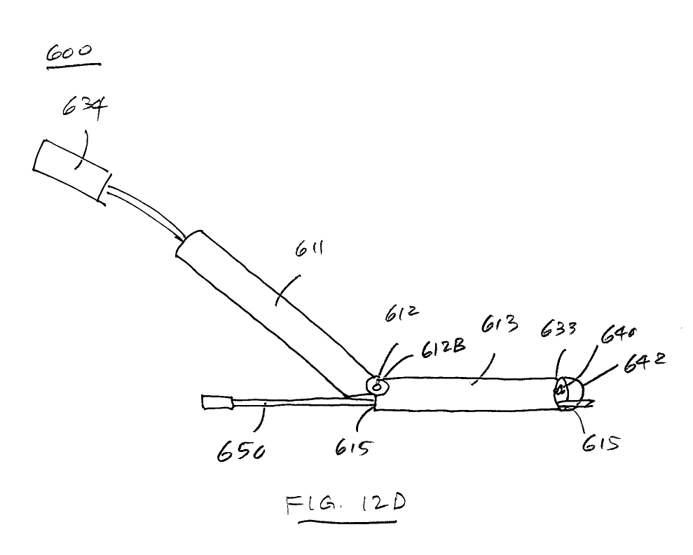

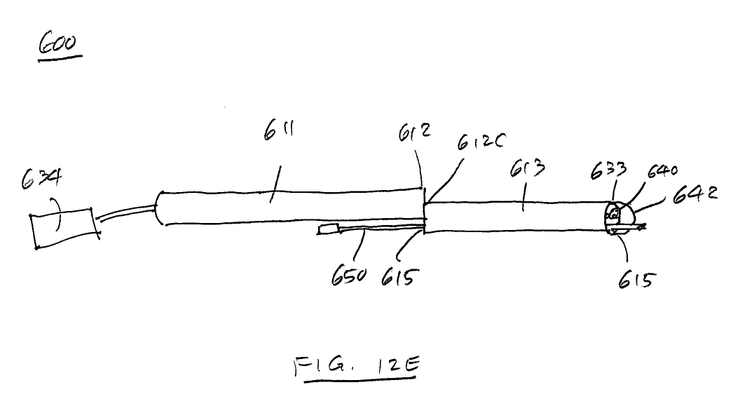

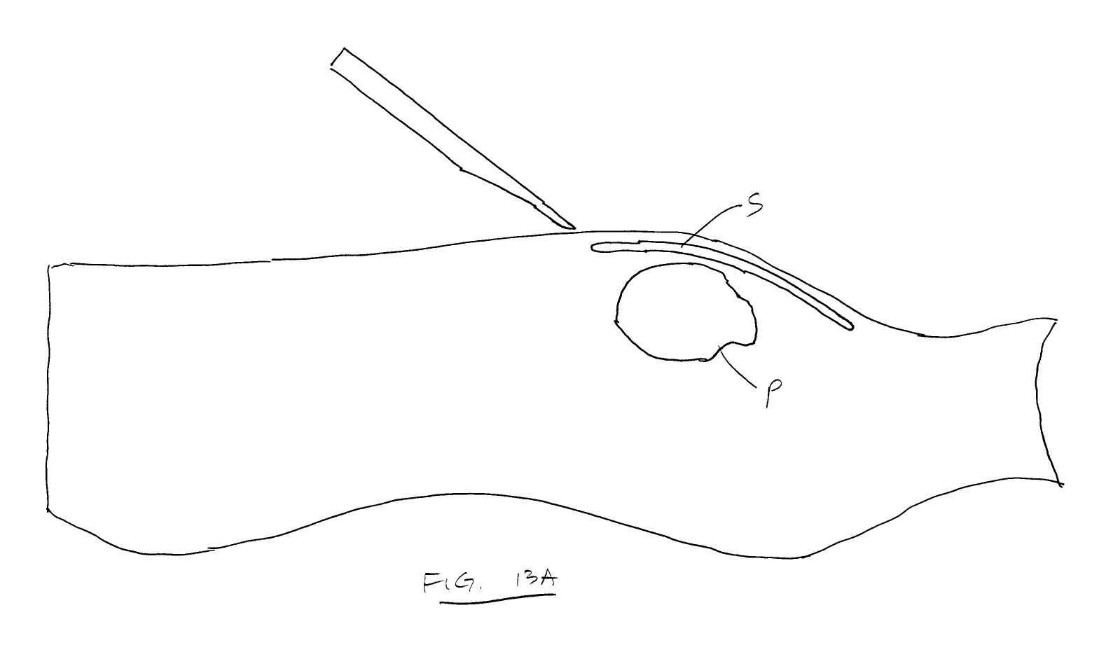

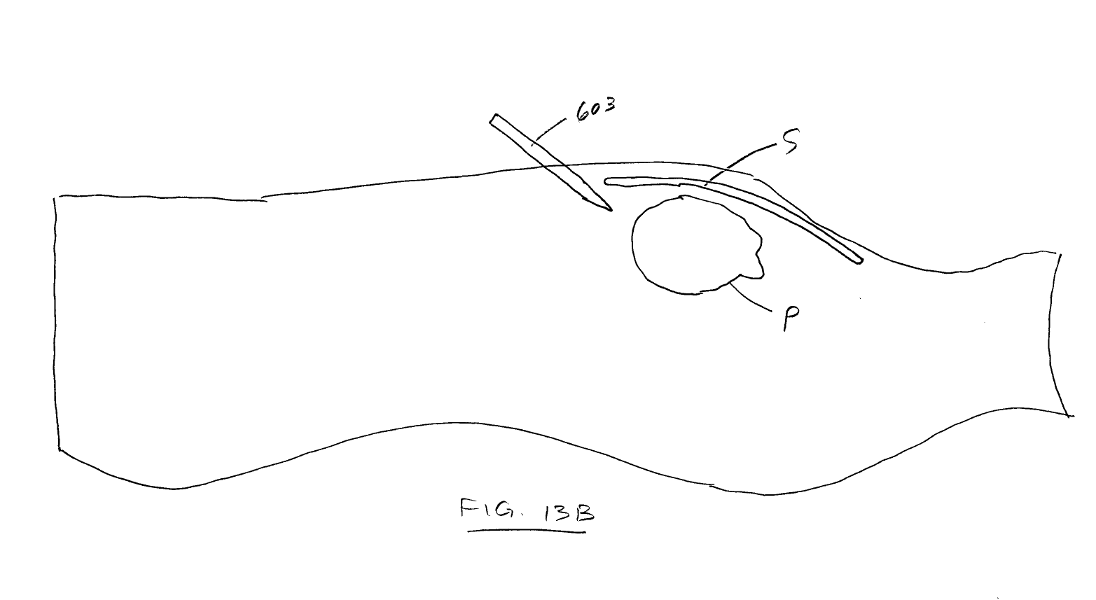

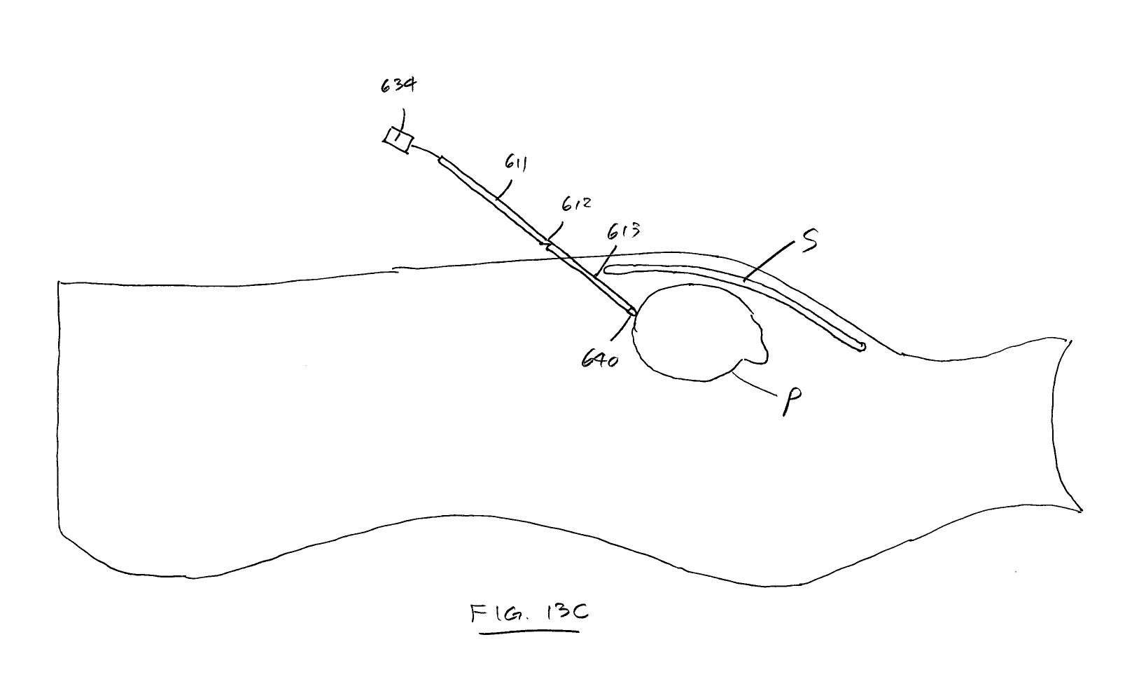

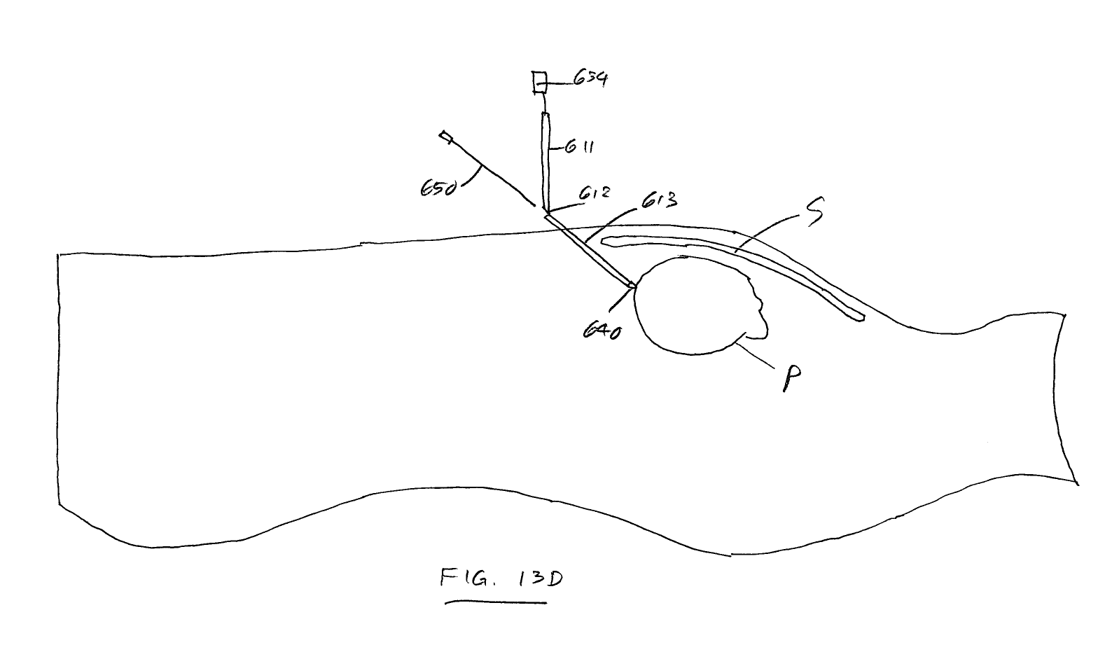

FIGS. 13A-13H show one method of using access device 600.

FIG. 13A shows a percutaneous puncture being made for a subxiphoid approach. Alternatively, an intercostal, apical, subclavian, suprasternal, or any other suitable approach may be used.

FIG. 13B shows a dilator 603 inserted through the puncture. Dilator 303 is used to dilate the puncture and then withdrawn.

FIG. 13C shows catheter 610 inserted through the puncture and distal end 633 of distal segment 613 positioned at or near the surface of the pericardium P. Visualization element 640 is used to guide distal segment 613 along the posterior aspect of the sternum S to the surface of the pericardium P.

FIG. 13D shows joint 612 opened to expose distal working lumen 615.

FIG. 13E shows a working element 650 passed through distal working lumen 615, and advanced through the pericardium P to create an access site. For a working element 650 having a needle tip 655, visualization element 640 is used to visualize working element 650 as it is advanced through pericardium P. For a working element 650 used with RF energy, visualization element 640 is used to visualize working element 650 as RF energy is passed through working element 650 to penetrate pericardium P. Working element 650 may be rotated so that a desired surface is visible to visualization element 640. Saline, contrast, medications, and/or other fluids may be introduced through working element 650 into the pericardial space.

FIG. 13F shows guidewire 605 advanced through working element 650 and positioned in the pericardial space.

FIG. 13G shows catheter 610 and working element 650 withdrawn, leaving guidewire 605 in place.

FIG. 13H shows a sheath 680 advanced over guidewire 605 through the puncture and the access site and into the pericardial space. Other devices or guidewires may be advanced through sheath 680 to access the pericardial space. Saline, contrast, medications, and/or other fluids may be introduced through sheath 680 into the pericardial space.

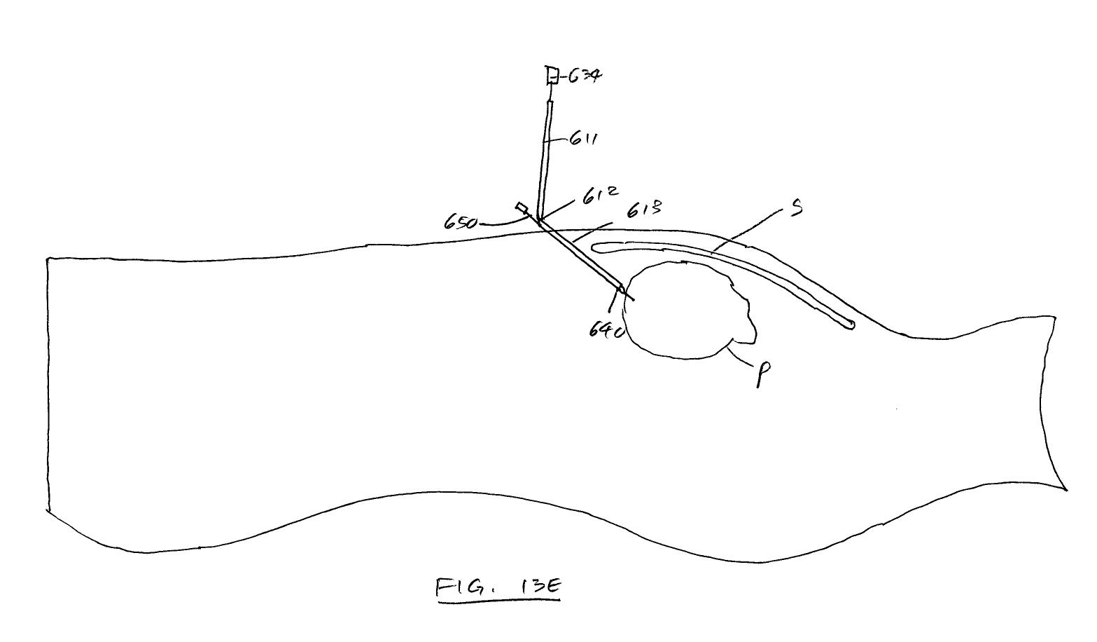

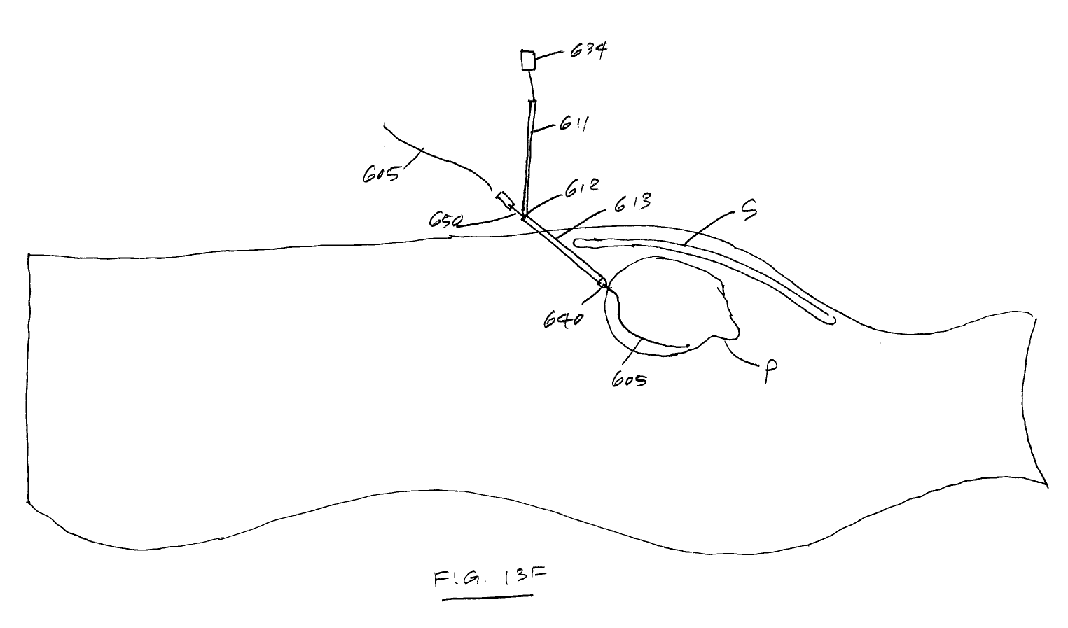

FIGS. 14A-14B show one embodiment of an imaging package 700. Imaging package 700 includes a imaging element 710, an alignment block 720, wires 730, and an adhesive 740.

Imaging element 710 includes an imaging chip 712 and an optical element 714. Imaging chip 712 may be a CCD, CMOS, or any other suitable imaging device. Imaging chip 712 may be coupled to a printed circuit board having on its back surface solder pads 713. Optical element 714 may be an infrared filter. Optical element 714 may also be a cover, a pinhole aperture, and/or one or more lenses.

Alignment block 720 includes a plurality of holes 722 which are aligned with the solder pads 713 of imaging chip 712. Alignment block 720 serves to facilitate the coupling of wires 730 to solder pads 713.

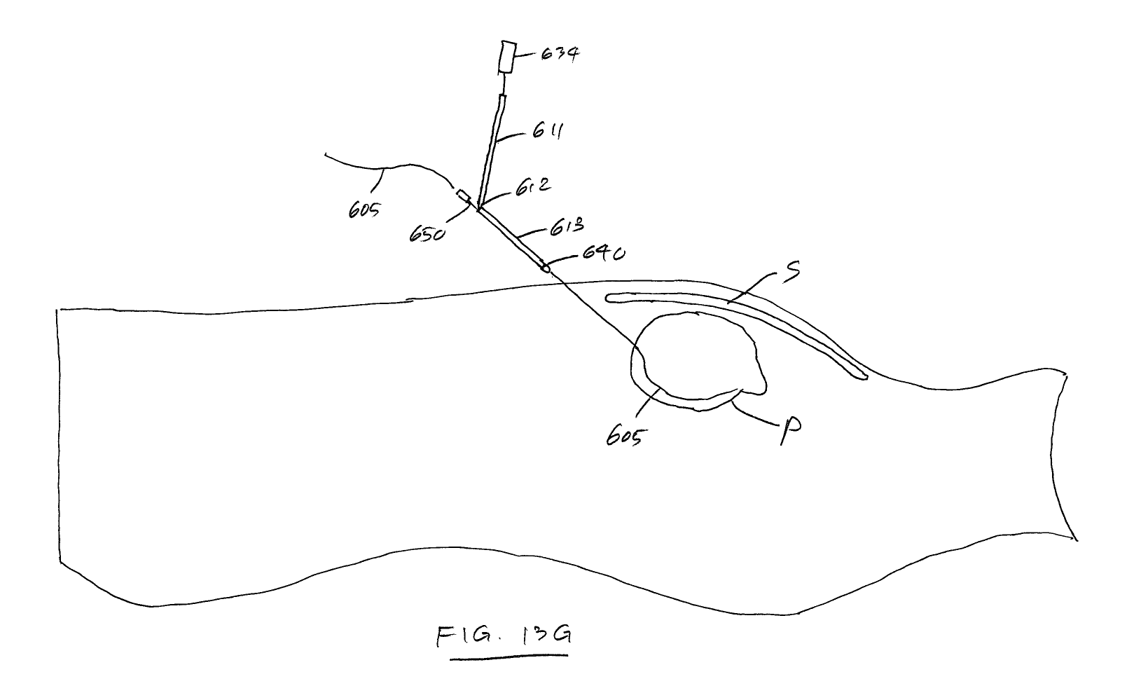

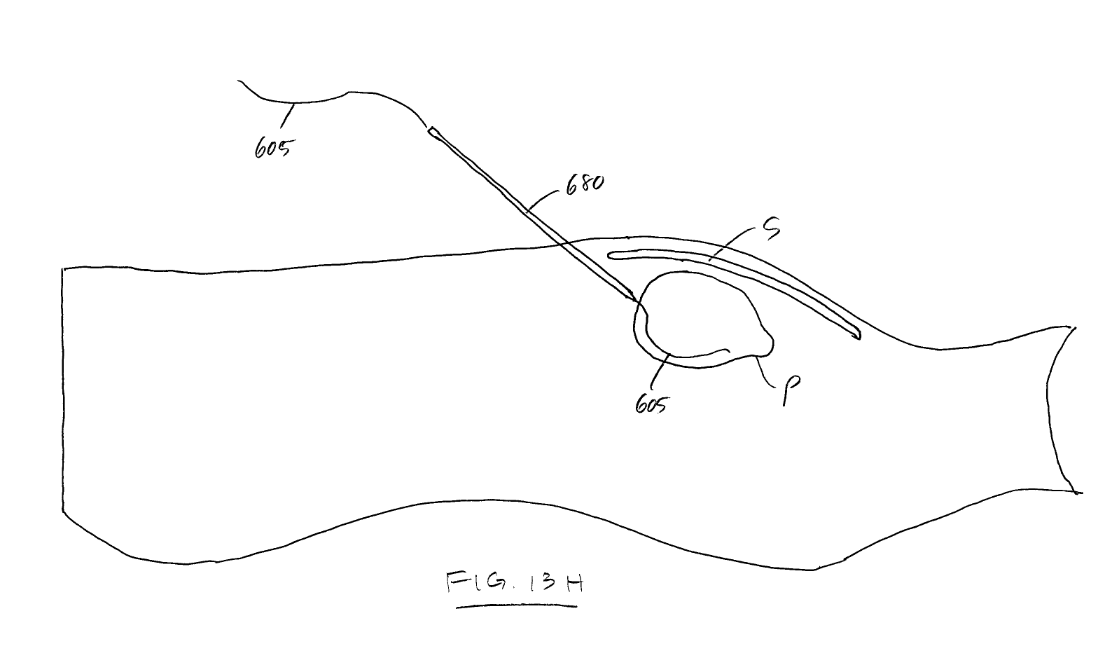

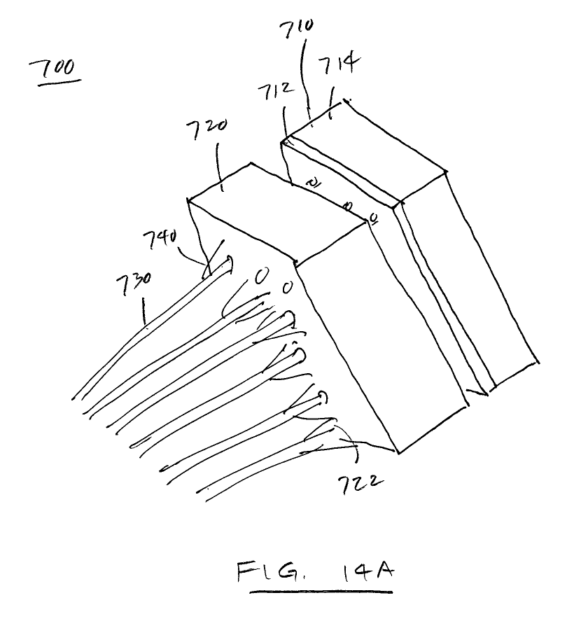

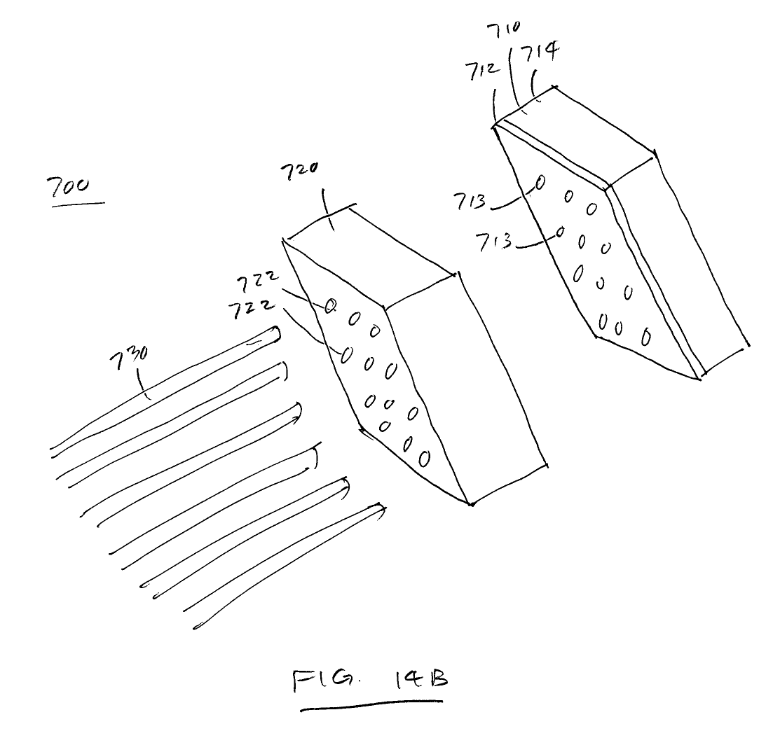

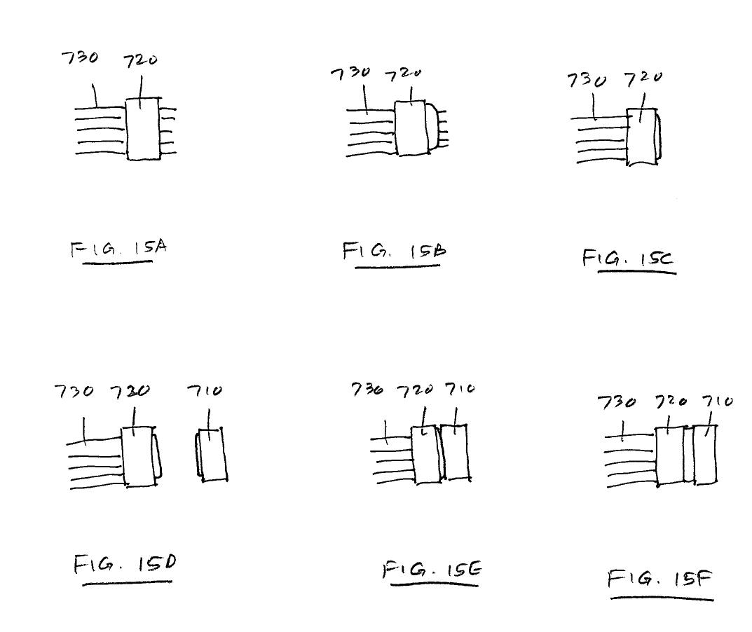

FIGS. 15A-15F show one method of assembling imaging package 700. FIG. 15A shows wires 730 passed through holes 722 of alignment block 720 so that wires 730 protrude from the other side of alignment block 720. FIG. 15B shows an adhesive such as epoxy applied to the protruding wires 730. Adhesive may also be applied to the insertion side. FIG. 15C shows the epoxy and protruding wires 730 machined or sanded down to a substantially smooth or even surface. Wires 730 protrude slightly from alignment block 720. FIG. 15D shows a conductive material such as a soft solder applied to the back side of imaging element 710. FIG. 15E shows imaging element 710 with the soft solder being brought into contact with the machined or sanded down surface of alignment block 720 and into contact with wires 730. FIG. 15F shows an adhesive such as epoxy applied in between imaging element 710 and alignment block 720 to secure imaging element 710 to alignment block 720.

Visualization element 140, 240, 340, 540, or 640 may include a CCD, CMOS, or any other suitable imaging device, such as those available Omnivision Technologies, Inc., Santa Clara, Calif. Alternatively, visualization element 140, 240, 340, 540, or 640 may include a fiber optic device. Visualization element 140, 240, 340, 540, or 640 may also be an IntroSpicio 120 CMOS camera, available from Medigus Ltd., Omer, Israel.

Although the above embodiments and methods describe using the access device to visualize and access the pericardial space, this device may be used to visualize and access any space, tissue, or organ in the body. Examples include the heart, peritoneum, diaphragm, mediastinal structures, thoracic, liver, kidney, thoracic, and abdominal regions.

While the foregoing has been with reference to particular embodiments of the invention, it will be appreciated by those skilled in the art that changes in these embodiments may be made without departing from the principles and spirit of the invention.

All publications and patent applications mentioned in this specification are herein incorporated by reference to the same extent as if each individual publication or patent application was specifically and individually indicated to be incorporated by reference.

1. An access device comprising:
a visualization catheter;
a visualization element coupled to a distal end of the visualization catheter;
an open track formed along a length of the visualization catheter;
a working catheter configured to slide along the track until a distal end of the working catheter is in a vicinity of the visualization element; and
a working lumen extending through the working catheter.

2. The device of claim 1, wherein the track is substantially semicircular.

3. The device of claim 2, wherein the working catheter is substantially circular.

4. The device of claim 1, further comprising a runner coupled the working catheter, the runner configured to slide at least partially within the track.

5. The device of claim 4, wherein the runner is the same length as the working catheter.

6. The device of claim 4, wherein the runner is shorter than the working catheter.

7. The device of claim 6, wherein the runner is located at a distal portion of the working catheter.

8. The device of claim 4, wherein the track and the runner are substantially circular.

9. The device of claim 4, wherein the track and the runner are T-shaped.

10. The device of claim 1, wherein the track includes a stop at the distal end of the visualization catheter which prevents the distal end of the working catheter from sliding beyond the distal end of the visualization catheter.

11. The device of claim 1, further comprising a handle coupled to a proximal end of the visualization catheter.

12. The device of claim 11, wherein the handle includes an opening for the working catheter, the opening configured to receive the working catheter and align the working catheter with the track.

13. The device of claim 1, wherein the visualization catheter is straight.

14. The device of claim 1, wherein the visualization catheter is curved.

15. The device of claim 1, further comprising a working element capable of being inserted through the working lumen.

16. The device of claim 15, wherein the working element is a needle.

17. A method of accessing a region inside a body, the method comprising:
inserting a visualization catheter into the body through a percutaneous puncture, the visualization catheter having an open track formed along its length;
locating the region inside the body with the aid of a visualization element coupled to a distal end of the visualization catheter; and
sliding a working catheter along the track through the percutaneous puncture until a distal end of the working catheter is in a vicinity of the visualization element; and
inserting a working element through a working lumen extending through the working catheter to access the region inside the body with the aid of the visualization element.

18. The method of claim 17, wherein the working catheter includes a runner configured to slide at least partially within the track, and wherein sliding the working catheter along the track includes sliding the runner at least partially within the track.

19. The method of claim 17, wherein sliding a working catheter along the track includes sliding the working catheter along the track until the working catheter reaches a stop at the distal end of the visualization catheter.

20. The method of claim 17, wherein sliding a working catheter along the track includes sliding the working catheter through an opening in a handle coupled to a proximal end of the visualization catheter, the opening configured to receive the working catheter and align the working catheter with the track.

21-39. (canceled)
